(19) United States
(12) Patent Application Publication
(10) Pub. No.: US 2008/0274950 A1
(43) Pub. Date: Nov. 6, 2008

(54) Cell Adhesion by Modified Cadherin Molecules

(75) Inventors: Peter Kilshaw, Cambridge (GB); Elaine Corps, Cambridge (GB); Oliver Harrison, South Yorkshire (GB)

Correspondence Address: PROSKAUER ROSE LLP, 1001 PENNSYLVANIA AVE, N.W.,, SUITE 400 SOUTH, WASHINGTON, DC, 20004, US

(73) Assignee: The Babraham Institute, Cambridge (GB)

(21) Appl. No.: 11/814,339

(22) PCT Filed: Jan. 18, 2006
(86) PCT No.: PCT/GB2006/000173

§ 371 Date: Apr. 15, 2008

(30) Foreign Application Priority Data
Jan. 21, 2005 (GB) 0501216.6
Jul. 27, 2005 (GB) 0515423.2

(51) Int. Cl. A61K 38/17; C07K 14/435; C12N 5/00; G01N 33/53; C07H 21/00; C12N 5/10

(52) U.S. Cl. 514/8; 530/395; 435/375; 435/4; 536/23.5; 435/325

(57) ABSTRACT

The potential role of so called ‘cell adhesion recognition motifs’ (CARs) in cadherin adhesion has been emphasized. Due to the importance of cadherin binding in biological process, there remains a need to develop effective ways of manipulating cadherin adhesion. According to the present invention, there is provided a pair of cadherin molecules modified to enhance intermolecular adhesion (i.e. adhesion or binding between the pair of cadherin molecules) compared with corresponding unmodified cadherin molecules. Intermolecular adhesion between the cadherin molecules may be enhanced by reducing or eliminating intramolecular binding within each cadherin molecule. For example, intramolecular binding may be reduced or eliminated by diminishing or preventing intramolecular binding of an N-terminal binding strand of each cadherin molecule with a binding strand acceptor pocket of each cadherin molecule. Additionally or alternatively, the intramolecular binding may be reduced or eliminated by diminishing or preventing the formation of an intramolecular ionic bond between the NH2 terminus of each cadherin molecule with a contact acidic amino acid residue of each cadherin molecule.

The present invention relates to cadherin molecule adhesion.

Cadherins are a family of cell surface adhesion molecules that are essential for maintaining the structural integrity of all vertebrate solid tissues. The cadherin family includes classical (“Type I”) cadherins, non-classical (“Type II”), desmosomal cadherins and protocadherins (reviewed in Patell et al., 2003). Cadherins determine cell-cell recognition during morphogenesis and have signalling functions which influence cell migration and differentiation (Cavallaro and Christofori, 2004; Hirano et al., 2003; Thiery, 2003; Wheelock and Johnson, 2003). Indeed, cadherins provide the principal adhesion mechanism for maintaining the integrity of all solid tissues and, in addition, play a major role in controlling segregation of cells during organ formation in embryonic development. Cadherins are often found to malfunction in cancer. In metastatic carcinomas, expression of E-cadherin is often down-regulated or the molecule has suffered a functional mutation. In contrast, N-cadherin is frequently upregulated and through its cell signalling capacity stimulates invasive behaviour. Lack of cadherin-mediated adhesion is a major cause of cancer metastasis.

Cadherin molecules usually stick to their own type, i.e. E-cadherin sticks to another E-cadherin molecule but not as well to an N-cadherin molecule. Cadherins engage each other at their tip ends and the interaction between individual molecules has a low affinity but, cumulatively, they provide strong adhesion between cells. Cadherin-cadherin contacts, as well as providing an ‘intercellular glue’, also convey signals to the cell and modulate signalling by growth factors. In the case of N-cadherin these signals promote cell survival and cell migration.

Adhesive interactions by cadherins are mostly, but not exclusively, homophilic and cadherin type-specific. Classical cadherins comprise five extracellular β-barrel-like domains (domains “EC1” to “EC5”, also known as “ectodomains”), a transmembrane domain and a cytoplasmic domain. Each of the extracellular domains contains seven β strands and, in most cases, calcium binding sites. Adhesion requires the presence of calcium bound in the interdomain junctions of the extracellular domains and it is known that this rigidifies the cadherin molecule into a curved rod-like structure projecting from the cell (Boggon et al., 2002; He et al., 2003; Miyaguchi, 2000; Pokutta et al., 1994). Despite more than a decade of research, the mechanism by which cadherin extracellular domains form adhesive contacts remains controversial.

Insights into the process of adhesion have come mainly from four experimental strategies: observations of the effects of point mutations or domain deletions on cell adhesion, co-immunoprecipitation of epitope-tagged cadherin molecules in adhesive complexes between cells, structural studies of cadherins by NMR or X-ray crystallography, and physical studies, including measurements of intermolecular forces between cadherin molecules and direct observation of cadherins by electron microscopy. Cumulatively, these techniques have led to several alternative models for adhesion.

Amino acids which co-ordinate calcium in the junction between the first and second domains, EC1 and EC2 (also known as “ECD1” and “ECD2”, respectively), have been shown to play an essential role in adhesion (Corps et al., 2001; Klingelhofer et al., 2002) and structural studies have suggested that calcium will instigate dimerisation of the recombinant protein EC1-EC2 via contact surfaces in the domain junction and EC1 (Haussinger et al., 2002; Pertz et al., 1999). This effect of calcium has been demonstrated by physical measurements and electron microscopy (Alattia et al., 1997). Scanning mutagenesis in the N-terminal domain (EC1) has shown that tryptophan 2 (Trp2), the second amino acid of the mature cadherin molecule, and amino acids lining an adjacent hydrophobic pocket are also indispensable for adhesion (Kitagawa et al., 2000; Tamura et al., 1998). The importance of Trp2 has been confirmed by immunoprecipitation studies which have demonstrated that this residue is required for the formation of both adhesive (trans) dimers and lateral (cis) dimers (Laur et al., 2002; Ozawa, 2002). A possible explanation for the significance of Trp2 has been provided by three X-ray crystallography studies which have revealed a mechanism for dimerisation in which Trp2 in strand A of EC1 docks into a hydrophobic pocket in EC1 of its neighbour, a mutual process which holds the two EC1 protomers together (Boggon et al., 2002; Haussinger et al., 2004; Shapiro et al., 1995). In principle this interaction (strand exchange) could mediate dimerisation in either cis- or trans-alignment. A recent immunoprecipitation study which was designed to discriminate between strand exchange and a calcium-mediated mechanism for dimerisation is consistent with the strand exchange model (Troyanovsky et al., 2003).

A different perspective has emerged from measurements of intermolecular forces between recombinant cadherin molecules. That data suggest that contact surfaces on two or more cadherin domains are required for adhesion and that opposing cadherin molecules can engage in several alternative anti-parallel alignments (Chappuis-Flament et al., 2001; Sivasankar et al., 2001; Zhu et al., 2003). That idea is at variance with direct observation, by electron microscopy, of purified recombinant cadherin molecules and cadherins in junctional complexes. Those images suggest that both cis- and transdimerisation takes place exclusively via EC1 (Ahrens et al., 2003; Ahrens et al., 2002; He et al., 2003; Pertz et al., 1999). A central issue in those conflicting models is whether Trp2 serves only to stabilise an adhesive contact surface in domain 1 or whether strand exchange is the primary event in adhesion.

The potential role of so called ‘cell adhesion recognition motifs’ (CARs) in cadherin adhesion has been emphasised. A principal CAR in cadherins is the amino acid sequence HAV in domain 1 (EC1). EC1 is the most N-terminal domain of a mature cadherin molecule obtained after the prodomain or precursor sequence of amino acids has been removed by normal cellular processing. Cyclic peptides which include the HAV sequence have been shown to inhibit cadherin-mediated adhesion and in some circumstances to trigger apoptosis. The potential use of HAV-type peptides as pharmaceutical agents to inhibit cell adhesion in a wide range of therapeutic applications or to stimulate cadherin-mediated signalling has been appreciated by companies such as Adherex Inc., Ottawa. Adherex patent documents cover many potential clinical applications for peptide mimetics of CARs, antibodies which recognise CARs or other CAR-binding agents. Their lead product, Exherin, is an HAV cyclic peptide which inhibits N-cadherin function.

Due to the importance of cadherin binding in biological process, there remains a need to develop effective ways of manipulating cadherin adhesion both for in vivo and potentially in vitro applications.

According to a first aspect of the present invention, there is provided a pair of cadherin molecules modified to enhance intermolecular adhesion (i.e. adhesion or binding between the pair of cadherin molecules) compared with corresponding unmodified cadherin molecules.

The present inventors show definitive evidence for the primary mechanism of cadherin-mediated adhesion. Our data (see below) shows that the so-called ‘strand-exchange’ model is correct. It is a further example of so called ‘3D domain swapping,’ one of several mechanisms that cause proteins to dimerise or polymerise. This mechanism does not depend on a cadherin CAR—we now have evidence that the primary and crucial molecular contact in cadherin-mediated adhesion does not involve HAV or any CAR—and is quite distinct from the idea which forms the scientific basis for the Adherex strategy. It is a novel and unexpected finding that cadherin molecules as modified herein have modulated adhesion (or altered adhesive) properties of the type disclosed. In particular, an increase in intermolecular adhesion between complementary pairs of cadherin molecules compared with that between normal cadherin molecules is novel and unpredicted. This modulating effect has several uses and benefits, as elaborated herein.

In the present invention, intermolecular adhesion between the cadherin molecules may be enhanced by reducing or eliminating intramolecular binding within each cadherin molecule. For example, intramolecular binding may be reduced or eliminated by diminishing or preventing intramolecular binding of an N-terminal binding strand of each cadherin molecule with a binding strand acceptor pocket of each cadherin molecule. For example, the N-terminal binding strand may be derived from or equivalent to the βA strand (with tryptophan at amino acid position 2) of the EC1 domain of mature wild-type human N-cadherin (or a function equivalent thereof; see below). For example, the binding strand acceptor pocket may be derived from or equivalent to the hydrophobic Trp 2 acceptor pocket in EC1 which accepts insertion of tryptophan at amino acid position 2 of mature human N-cadherin (or a function equivalent thereof; see below). Intramolecular binding may be prevented or eliminated or diminished by substituting Trp2 with an alternative amino acid, for example glycine, and/or by obstructing the hydrophobic Trp 2 acceptor pocket, for example by introducing the mutation Ala80Ile (with reference to alanine at amino acid position 80 of mature wild-type human N-cadherin or a functional equivalent thereof see below).

Additionally or alternatively, the intramolecular binding may be reduced or eliminated by diminishing or preventing the formation of an intramolecular ionic bond (for example, a salt bridge) between the NH₂ terminus of each cadherin molecule with a contact acidic amino acid residue (for example, glutamic acid, aspartate, asparagine or glutamine) of each cadherin molecule. The contact acidic amino acid residue may, for example, be derived from or equivalent to glutamic acid at amino acid position 89 of mature N-cadherin (or a function equivalent thereof; see below).

In accordance with the findings of the present inventors, intermolecular adhesion may be facilitated by an ionic bond between a contact acidic amino acid residue of one cadherin molecule and the NH₂ terminus of the other cadherin molecule. Intermolecular adhesion may also be facilitated by binding of an N-terminal binding strand of one cadherin molecule with a binding strand acceptor domain of the other cadherin molecule. The features of the cadherin molecules contributing to intermolecular adhesion are as mentioned herein for intramolecular binding.

As used herein, “intermolecular adhesion” means adhesion or binding between two (or more) cadherin molecules. Intermolecular adhesion may include insertion or “docking” of the N-terminal binding strand of a first cadherin molecule with a binding strand acceptor pocket of a second cadherin molecule (for example the docking or insertion of Trp2 of a first mature N-cadherin molecule or a modified version thereof into the hydrophobic Trp2 acceptor pocket in the EC1 domain of a second mature N-cadherin molecule or a modified version thereof), and/or formation of an intermolecular ionic bond between NH₂ terminus of a first cadherin molecule and the contact amino acid residue of a second cadherin molecule (for example, the formation of a salt bridge between the NH₂ terminus of a first mature N-cadherin molecule or modified version thereof and Glu89 of a second mature N-cadherin molecule or a modified version thereof).

As used herein, “intramolecular binding” means binding (or self-docking or adhesion) within a cadherin molecule to form a closed or partially closed monomeric cadherin molecule. Intramolecular binding may include insertion or “docking” of the N-terminal binding strand of each cadherin molecule with a binding strand acceptor pocket of each cadherin molecule (for example the docking or insertion of Trp2 into the hydrophobic Trp2 acceptor pocket in the EC1 domain of mature wild-type human N-cadherin or a modified version thereof) and/or formation of an intramolecular ionic bond between NH₂ terminus and the contact amino acid residue of the cadherin molecule (for example, the formation of a salt bridge between the NH₂ terminus and Glu89 of mature wild-type human N-cadherin or a modified version thereof).

The present invention is based, in part, on the finding that cadherin adhesion depends on a dynamic equilibrium between intramolecular binding and intermolecular adhesion. The dynamic equilibrium means that structural features which bring about adhesion can be manipulated to favour intramolecular binding or intermolecular adhesion. These structural features include the NH₂ terminus, the contact amino acid residue, the N-terminal binding strand and the binding strand acceptor pocket, of each cadherin molecule (or polypeptide). Intramolecular binding occurs when the N-terminal binding strand on one cadherin molecule binds with the binding strand acceptor pocket of the same molecule, a reaction that is stabilised by the formation of an ionic bond (for example, a salt bridge) between the NH₂ terminus of the cadherin molecule and the contact amino acid residue of the same molecule. Intermolecular adhesion occurs when the NH₂ terminus of a first cadherin molecule forms an ionic bond (for example, a salt bridge) with the contact amino acid residue of a second cadherin molecule, and/or when the N-terminal binding strand on the first cadherin molecule binds with the binding strand acceptor pocket of the second cadherin molecule.

In one aspect of the present invention, the cadherin molecules may be modified by altering the primary structure of each cadherin molecule.

For example, the following pairs of cadherin molecules may be used according to the present invention:

(i) a first cadherin molecule in which the N-terminus is extended by addition of one or more amino acids to a mature (processed) cadherin molecule (for example mature N-cadherin), and/or in which the correct processing of the cadherin prodomain or precursor sequence has been prevented, in each case preventing the formation of an intramolecular ionic bond; and a second cadherin molecule in which the acidic acid residue is mutated to remove functionality, thereby preventing formation of an intramolecular ionic bond (for example, Glu89 of mature N-cadherin mutated to Ala89), and/or in which binding of the N-terminal binding strand of one cadherin molecule (for example the βA strand of mature N-cadherin with tryptophan at amino acid position 2) is prevented from binding into the binding strand acceptor pocket (for example, by mutation of alanine at amino acid position 80 of mature N-cadherin to isoleucine, or an equivalent mutation, to block tryptophan docking into the hydrophobic acceptor pocket); and
(ii) a first cadherin molecule in which the N-terminal binding strand of one cadherin molecule (for example the EC1 domain βA strand of mature N-cadherin with tryptophan at amino acid position 2) has been functionally mutated, for example by removal or replacement of tryptophan at amino acid position 2 of mature N-cadherin; and a second cadherin molecule as the second cadherin molecule in (i) above.

Additionally or alternatively, the cadherin molecules may, for example, be modified by one or more mutations to the βA strand of domain EC1 which remove, add or substitute one or more amino acids so as to inhibit or diminish intramolecular binding and/or to enhance or facilitate intermolecular adhesion.

Additionally or alternatively, the cadherin molecules may be modified by contacting one or both cadherin molecules with one or more substances which enhance intermolecular adhesion and/or reduce or eliminate intramolecular binding. The substance may be an organic molecule, preferably a small organic molecule, a drug, a peptide, a peptidometic, an antibody and/or a modified cadherin molecule that contacts each cadherin molecule. For example, the substance may bind to the cadherin molecules such that intramolecular binding is reduced or inhibited by diminishing or preventing the formation of the intramolecular ionic bond between the NH₂ terminus of each cadherin molecule with the contact acidic amino acid residue of each cadherin molecule, and/or by preventing or diminishing intramolecular binding of an N-terminal binding strand of each cadherin molecule with a binding strand acceptor pocket of each cadherin molecule, such that intermolecular adhesion is enhanced.

Further provided according to the present invention is a pair of polypeptides which adhere to each other with an affinity greater than that between wild-type human N-cadherin molecules. Here, the polypeptides may be chemically synthesised using techniques well known to the person skilled in the art, for example. The structure of the polypeptides may be based on cadherin molecules (or functional fragments thereof) with desired intermolecular adhesion properties, for example the mutated cadherin molecules as described herein.

Also provided according to the present invention is a method of adhering a pair of polypeptides such as cadherin molecules by intermolecular adhesion, comprising contacting the polypeptides or cadherin molecules as defined herein, thereby allowing intermolecular adhesion.

Further provided is a method of increasing adhesion between two cadherin molecules, comprising reducing or eliminating intramolecular binding within each cadherin molecule and allowing formation of an ionic bond between an acidic amino acid residue of one cadherin molecule and the NH₂ terminus of the other cadherin molecule. Here, intermolecular adhesion may be facilitated by binding of an N-terminal binding strand of one cadherin molecule with a binding strand acceptor domain of the other cadherin molecule.

In a further aspect of the invention there is provided a substance (see above) which modulates intramolecular binding of one or more cadherin molecules by reducing or enhancing intermolecular adhesion between the molecules, wherein the substance excludes antibodies.

Also provided according to the present invention is the use of a substance (including antibodies and substances elaborated above) which modulates intramolecular binding of one or more cadherin molecules in order to modulate intermolecular adhesion between the molecules.

In another aspect there is provided a method for screening a candidate compound for the ability to modulate cadherin-mediated cell adhesion, comprising contacting the pair of cadherin molecules or the pair of polypeptides as defined herein in the presence and absence of the candidate compound and thereby evaluating the ability of the candidate compound to modulate cadherin-mediated cell adhesion.

There is also provided a method of increasing adhesion between a first cell and a second cell, comprising contacting the pair of cadherin molecules or the pair of polypeptides as defined herein, when one of the pair is attached to the first cell and the other of the pair is attached to the second cell.

The invention further provides an isolated nucleic acid molecule encoding the pair of cadherin molecules or the pair of polypeptides as defined herein. Alternatively, the invention provides a pair of isolated nucleic acid molecules in which each nucleic acid molecule encodes one of the pair of cadherin molecules or one the pair of polypeptides as defined herein. Also provided is an isolated nucleic acid molecule which hybridises under low stringent conditions, moderately stringent conditions or highly stringent conditions with the above-mentioned isolated nucleic acid molecule. The phrase “low stringency conditions” as used herein refers to hybridisation in 10% formamide, 5× Denhart's solution, 6×SSPE, 0.2% SDS at 42° C., followed by washing in 1×SSPE, 0.2% SDS, at 50° C. As used herein, the phrase “moderately stringent conditions” refer to conditions that permit target-DNA to bind a complementary nucleic acid that has about 60% identity, preferably about 75% identity, more preferably about 85% identity to the target DNA, with greater than about 90% identity to target-DNA being especially preferred. Preferably, moderately stringent conditions are conditions equivalent to hybridisation in 50% formamide, 5× Denhart's solution, 5×SSPE, 0.2% SDS at 42° C., followed by washing in 0.2×SSPE, 0.2% SDS, at 42° C. The phrase “high stringency conditions” are conditions that permit hybridisation of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridisation in 50% formamide, 5× Denhart's solution, 5×SSPE, 0.2% SDS at 42° C., followed by washing in 0.1×SSPE, and 0.1% SDS at 65° C. For solutions and methods, see for example Sambrook et al. (1989).

In another aspect of the invention there is provided a host cell comprising any of the group consisting of: the cadherin molecules (for example, one or both of the pair of cadherin molecules), the polypeptides (for example, one or both of the pair of polypeptides), the isolated nucleic acid molecule(s), and one or both of the pair of isolated nucleic acid molecules, as defined herein.

In alternative aspects of the invention, there is provided a pair of cadherin molecules modified to reduce or eliminate intermolecular adhesion compared with corresponding unmodified cadherin molecules, a pair of polypeptides which adhere to each other with an affinity lower than that between wild-type human N-cadherin molecules, a method of decreasing adhesion between tow cadherin molecules, and a method of decreasing adhesion between a first cell and a second cell. In these alternative aspects, the strategy as outlined for increasing adhesion is reversed, so as to favour intramolecular binding within the respective cadherin molecules or polypeptides.

The invention further provides a kit comprising any of the group consisting of: the cadherin molecules (for example, the pair of cadherin molecules), the polypeptides (for example, the pair of polypeptides), the isolated nucleic acid molecule(s), the pair of isolated nucleic acid molecules, and the host cell, as defined herein.

There is also provided according to the present invention a method for adhering two cadherin molecules, comprising the steps of:

(i) providing a first cadherin molecule with a binding domain comprising an N-terminus and a bridge amino acid residue (or “contact acidic amino acid residue” as described herein) at a site remote from the N-terminus and corresponding to residue Glu89 of mature wild-type human N-cadherin, in which the N-terminus of the first cadherin molecule is disrupted or prevented from forming an intramolecular ionic bond with the bridge amino acid of the first cadherin molecule;
(ii) providing a second cadherin molecule with a binding domain comprising an N-terminus and a bridge amino acid residue at a site remote from the N-terminus and corresponding to residue Glu89 of mature wild-type human N-cadherin, in which the bridge amino acid of the second cadherin molecule is disrupted or prevented from forming an intramolecular ionic bond with the N-terminus of the second cadherin molecule; and
(iii) contacting the first and second cadherin molecules.

Also provided is a method for adhering two cadherin molecules, comprising the steps of:

(i) providing a first cadherin molecule with a binding domain (or “N-terminal binding strand” as described herein) comprising a ligand amino acid residue corresponding to residue Trp2 of mature wild-type human N-cadherin and a ligand-acceptor hydrophobic pocket (or “binding strand acceptor pocket” as described herein) corresponding to the Trp2-acceptor hydrophobic pocket of mature wild-type human N-cadherin, in which the ligand amino acid of the first cadherin molecule is disrupted or prevented from intramolecular docking between (or into) the ligand-acceptor hydrophobic pocket of the first cadherin molecule;
(ii) providing a second cadherin molecule with a binding domain comprising a ligand amino acid residue corresponding to residue Trp2 of mature wild-type human N-cadherin and a ligand-acceptor hydrophobic pocket corresponding to the Trp2-acceptor hydrophobic pocket of mature wild-type human N-cadherin, in which the ligand amino acid of the second cadherin molecule is disrupted or prevented from intramolecular docking between (or into) the ligand-acceptor hydrophobic pocket of the second cadherin molecule; and
(iii) contacting the first and second cadherin molecules.

Also provided is a method for adhering two cadherin molecules, comprising the steps of:

(i) providing a first cadherin molecule with a binding domain which is disrupted or prevented from forming an intramolecular ionic bond between an N-terminus of the first cadherin molecule and a bridge amino acid residue of the first cadherin molecule at a site remote from the N-terminus and corresponding to residue Glu89 of mature wild-type human N-cadherin;
(ii) providing a second cadherin molecule with a binding domain which is disrupted or prevented from intramolecular docking between (or of) a ligand amino acid residue of the second cadherin molecule corresponding to residue Trp2 of mature wild-type human N-cadherin and (or into) a ligand-acceptor hydrophobic pocket of the second cadherin molecule corresponding to the Trp2-acceptor hydrophobic pocket of mature wild-type human N-cadherin; and
(iii) contacting the first and second cadherin molecules.

Step (iii) of the above methods may allow formation of an intermolecular ionic bond between a bridge amino acid residue on one cadherin molecule at a site remote from an N-terminus corresponding to residue Glu89 of mature wild-type human N-cadherin of the cadherin molecule and an N-terminus of the other cadherin molecule. Step (iii) of the above methods may additionally or alternatively allow intermolecular docking between a ligand amino acid on one cadherin molecule corresponding to residue Trp2 of mature wild-type human N-cadherin and a ligand-acceptor hydrophobic pocket of the other cadherin molecule corresponding to the Trp2-acceptor hydrophobic pocket of mature wild-type human N-cadherin.

Adhesion between the two cadherin molecules may be increased, for example compared to adhesion between two mature wild-type human N-cadherin molecules.

The intramolecular ionic bond may comprise or consist of a salt bridge.

The N-terminus and/or bridge amino acid may be prevented or disrupted from forming an intramolecular ionic bond by one or more of the following: an N-terminal extension of the binding domain from a distal amino acid residue corresponding to residue Asp1 of mature wild-type human N-cadherin (for example, an N-terminal extension comprising the amino acids GG or MDP); a molecule (for example a peptide, a peptidometic or an antibody) which binds to or near the distal amino acid residue; a molecule (for example a peptide, a peptidometic or an antibody) which binds to or near the bridge amino acid residue; and a functional mutation in the bridge amino acid residue (for example E89A).

The ligand amino acid residue and/or ligand-acceptor hydrophobic pocket may be disrupted or prevented from intramolecular docking by one or more of the following: a functional mutation in the ligand amino acid residue (for example W2G); a molecule (for example a peptide, a peptidometic or an antibody) which binds to or near the ligand amino acid residue; a molecule (for example an antibody, a peptide or a peptidometic) which binds to or near the ligand-acceptor hydrophobic pocket; a functional mutation in the ligand-acceptor hydrophobic pocket (for example A80I); and a peptide, peptidometic, drug or antibody which binds at or near to the base of the βA strand (or “N-terminal binding strand”) of one cadherin molecule.

Also provided is the use of the first cadherin molecule as defined above for binding to the second cadherin molecule as defined above, comprising contacting the first cadherin molecule with the second molecule.

The present invention provides in a further aspect a method of modifying the binding domain of a first cadherin molecule to modulate its binding with a complementary binding domain of a second cadherin molecule by ablating or reducing intramolecular docking within the binding domain and complementary binding domain, thereby making the binding domain of the first cadherin molecule available for intermolecular binding with the complementary binding domain of the second cadherin molecule, for example making the βA strand (or “N-terminal binding strand”) of the binding domain of the first cadherin molecule available for intermolecular binding with the complementary binding domain of the second cadherin molecule.

As used herein, the βA strand of cadherin is a strand corresponding to approximately the first ten to twelve N-terminal amino acid residues of mature wild-type cadherins, or a functional homologue thereof. In a preferred embodiment, the “N-terminal binding strand” of the cadherin molecule as described herein comprises or consists of the βA strand of cadherin. For mature wild-type human N-cadherin and most classical cadherins, Trp2 forms the second amino acid in the βA strand from the N terminus.

The invention also provides a method for modulating (for example, increasing) adhesion between two or more cadherin molecules comprising the step of contacting a first cadherin molecule with a complementary second cadherin molecule such that the N-terminus of only one of the cadherin molecules forms an intermolecular ionic bond (such as a salt bridge) with an amino acid residue (preferably an acidic amino acid residue) corresponding to residue Glu89 of mature wild-type human N-cadherin on the other cadherin molecule or an alternative acidic amino acid in the immediate vicinity (for example, within 1, 2, 3, 4, 5, 6, 10 or more amino acids from Glu89). Furthermore, an intermolecular bond between a ligand amino acid residue corresponding to residue Trp2 of mature wild-type human N-cadherin on one cadherin molecule may be formed with a ligand-acceptor hydrophobic pocket corresponding to the Trp2-acceptor hydrophobic pocket of mature wild-type human N-cadherin on the other cadherin molecule.

Also provided is a pair of cadherin molecules with complementary extracellular domains, wherein the N-terminus of only one of the cadherin molecules forms an intermolecular ionic bond (such as a salt bridge) with an amino acid residue (preferably an acidic amino acid residue) on the other cadherin molecule corresponding to residue Glu89 of mature wild-type human N-cadherin when the molecules are contacted. The pair of cadherin molecules when contacted may also form an intermolecular bond between a ligand amino acid residue corresponding to residue Trp2 of mature wild-type human N-cadherin on one cadherin molecule with a ligand-acceptor hydrophobic pocket corresponding to the Trp2-acceptor hydrophobic pocket of mature wild-type human N-cadherin on the other cadherin molecule.

In one embodiment of the invention, the base (or hinge) of a βA strand (or “N-terminal binding strand”) of a cadherin molecule is modified to place the βA strand into a position where intramolecular binding (or adhesion) is prohibited or reduced. The βA strand (or “N-terminal binding strand”) of the cadherin molecule may thus be able to bind to a complementary cadherin molecule by intermolecular binding (such as an intermolecular ionic bond and/or a ligand-acceptor hydrophobic bond of the type herein described). The molecule may be modified for example by a drug, a peptide, peptidometic or an antibody which targets the base of the βA strand (or “N-terminal binding strand”) or by substituting one or more amino acids in the βA strand with alternative amino acids or by adding or removing one or more amino acids from the βA strand without substitution. A modified cadherin molecule or pair of modified cadherin molecules thus formed are also within the scope of the present invention.

The βA strand (or “N-terminal binding strand”) of each of two cadherin molecules may form an intermolecular ionic bond (for example, a salt bridge) of the type herein described.

The invention encompasses the situation in which intramolecular binding or binding of the βA strand (or “N-terminal binding strand”) of each of two cadherin molecules is prevented while their intermolecular interaction is permitted.

The cell adhesion modulating agent of the present invention may be one or more cadherin molecules or polypeptide as described herein, or an agent that effects increased or decreased adhesion of cadherin molecules as described herein (for example, the substance as described herein). The cell adhesion modulating agent may also be a candidate compound detected by the method as described herein.

In another aspect of the invention, there is provided a method of stabilising adhesion between two cadherin molecules, comprising forming one or more thiol (for example, disulphide) bonds between amino acid residues, preferably cysteine residues, which are in close apposition during cadherin adhesion. For example, each cadherin molecule may comprise cysteine residues corresponding to amino acid positions 1 and/or 27 of wild-type mature human N-cadherin, which may be achieved for example by mutating Asp1 and Asp27 to Cys1 and Cys27, respectively. The invention also covers one or a pair of cadherin molecules comprising a structure modified for stabilising according to the above method.

The cadherin molecule or polypeptide of the present invention may comprise a modified mature wild-type human classical (type I) cadherin (for example N-cadherin, R-cadherin, E-cadherin, C-cadherin, P-cadherin, M-cadherin or T-cadherin), non-classical (type II) cadherin (for example VE-cadherin), desmosomal cadherin (for example desmocollin-1, desmocollin-2, desmocollin-3, desmoglein-1, desmoglein-2 or desmoglein-3), or protocadherin, or a functional homologue or functional fragment thereof (for example, modified extracellular domains 1 and 2 of wild-type human N-cadherin).

Each of or both cadherin molecules or polypeptides may comprise a modified N-cadherin-Fc fusion protein.

Each of or both cadherin molecules or polypeptides may be attached to one or more or the following: a cell; a surface (for example an assay surface such as a plastic plate); a magnetic bead; a non-magnetic bead; a solid matrix; and a semi-solid matrix.

Mature wild-type human N-cadherin has the amino acid sequence:SEQ ID No 1

D WVIPPINLPE NSRGPFPQEL VRIRSDRDKN LSLRYSVTGP
GADQPPTGIF IINPISGQLS VTKPLDREQI ARFHLRAHAV
DINGNQVENP IDIVINVIDM NDNRPEFLHQ VWNGTVPEGS
KPGTYVMTVT IADADDPNAL NGMLRYRIVS QAPSTPSPNM
FTINNETGDI ITVAAGLDRE KVQQYTLIIQ ATDMEGNPTY
GLSNTATAVI TVTDVNDNPP EFTAMTFYGE VPENRVDIIV
ANLTVTDKDQ PHTPAWNAVY RISGGDPTGR FAIQTDPNSN
DGLVTVVKPI DFETNRMFVL TVAAENQVPL AKGIQHPPQS
TATVSVTVID VNENPYFAPN PKIIRQEEGL HAGTMLTTFT
AQDPDRYMQQ NIRYTKLSDP ANWLKIDPVN GQITTIAVLD
RESPNVKNNI YNATFLASDN GIPPMSGTGT LQIYLLDIND
NAPQVLPQEA ETCETPDPNS INITALDYDI DPNAGPFAFD
LPLSPVTIKR NWTITRLNGD FAQLNLKIKF LEAGIYEVPI
IITDSGNPPK SNISILRVKV CQCDSNGDCT DVDRIVGAGL
GTGAIIAILL CIIILLILVL MFVVWMKRRD KERQAKQLLI
DPEDDVRDNI LKYDEEGGGE EDQDYDLSQL QQPDTVEPDA
IKPVGIRRMD ERPIHAEPQY PVRSAAPHPG DIGDFINEGL
KAADNDPTAP PYDSLLVFDY EGSGSTAGSL SSLNSSSSGG
EQDYDYLNDW GPRFKKLADM YGGGDD.

The mature wild-type human N-cadherin sequence corresponds to residues 160 to 906 of precursor Neural-cadherin (N-cadherin) provided in Genbank accession No. P19022.

As used herein, unless otherwise stated, the term “cadherin” or “cadherin molecule” encompasses a functional fragment, homologue or variant thereof. The terms “cadherin molecule(s)” or “polypeptide(s)” as used in the present invention also include within their scope a functional fragment, equivalent, homologue or variant of wild-type mature human N-cadherin (see below). Each cadherin molecule or each polypeptide may have at least 20, 25, 30, 35, 40, 45, 50, 55, 60, 65, 70, 75, 80, 85, 90, 95% or greater homology with wild-type mature human N-cadherin or a functional fragment, equivalent, homologue or variant of wild-type human N-cadherin. Alternatively, each cadherin molecule or each polypeptide may have at least 20, 25, 30, 35, 40, 45, 50, 55, 60, 65, 70, 75 or 80, 85, 90, 95% or greater homology with wild-type mature human N-cadherin or a functional fragment, equivalent, homologue or variant of wild-type human N-cadherin excluding the transmembrane and/or cytoplasmic domains of wild-type human N-cadherin.

In specific embodiments described below, the extracellular region (or “ectodomain”) of N-cadherin is fused to human IgG Fc to form a N-cadherin-Fc fragment which is used in binding assays. In such specific embodiments, it is the cadherin ectodomain which exhibits binding function. The term “cadherin” or “cadherin molecule” may thus encompass a molecule (for example, a polypeptide) comprising the cadherin ectodomain but lacking other cadherin domains. Similarly, the term “cadherin” or “cadherin molecule” encompasses a molecule (for example, a polypeptide) which is a functional fragment, homologue or variant of at least one EC domain of a cadherin molecule. The molecule or polypeptide in one embodiment comprises the EC1 and EC2 domains of cadherin (for example, of mature human N-cadherin as defined above) but lacks other cadherin domains.

### Medical and Therapeutic Applications of the Invention

The invention provides in one aspect a method for enhancing delivery of a drug to a cell (for example a tumour cell or a central nervous system cell), comprising administering to the cell: (a) a cell adhesion modulating agent as described herein that modulates cadherin-mediated cell adhesion; and (b) a drug; and thereby enhancing the delivery of the drug to the cell.

Also provided according to the invention is a method for inhibiting the development of cancer in an animal (for example a mammal such as a human), comprising administering to the animal a cell adhesion modulating agent as described herein that modulates cadherin-mediated cell adhesion, thereby inhibiting development of cancer in the animal. A cell adhesion modulating agent which functions as an anticancer compound is further provided according to present invention. Such anticancer compounds may be used for example in combination with classical anti-cancer therapy (for example, irradiation) to prevent tumour cells from migration.

In another aspect there is provided a method for inhibiting angiogenesis in an animal (for example a mammal such as a human), comprising administering to the animal a cell adhesion modulating agent as described herein that modulates cadherin-mediated cell adhesion, thereby inhibiting angiogenesis in the animal. Angiogenesis is the growth of blood vessels that occur in both normal and diseased cells. In cancer cells there is uncontrolled cellular growth sustained by hyper active angiogenesis. Therapeutics that can specifically target the blood supply of cancer cells are known as angiolytics and have been widely studied since the 1990s which led to the first approved anti-angiogenic, Avastin, appearing in early 2004. Angiolytic drugs are also known as vascular targeting agents (“VTAs”) and are a new class of drug designed to cause structural damage to the cells of blood vessels which in turn limits blood flow to a vascularised tumour cell causing cell death. The present invention allows for the process of angiogenesis to be inhibited by using a cell adhesion modulating agent as described herein that modulates cadherin-mediated cell adhesion.

In a further aspect there is provided a method for enhancing wound healing in an animal (for example a mammal such as a human), comprising administering to the animal a cell adhesion modulating agent as described herein that modulates cadherin-mediated cell adhesion, thereby enhancing wound healing in the animal.

The invention further provides a method for enhancing adhesion of foreign tissue implanted within an animal (for example a mammal such as a human), comprising administering to the animal a cell adhesion modulating agent as described herein that modulates cadherin-mediated cell adhesion, thereby enhancing adhesion of foreign tissue implanted within the animal.

Also provided is a method for modulating the immune system of an animal (for example a mammal such as a human), comprising administering to the animal a cell adhesion modulating agent as described herein that modulates cadherin-mediated cell adhesion, thereby modulating the immune system of the animal.

In another aspect, there is provided a method for modulating vasopermeability in an animal (for example a mammal such as a human), comprising administering to the animal a cell adhesion modulating agent as described herein that modulates cadherin-mediated cell adhesion, thereby modulating vasopermeability in the animal.

Also provided is a method for treating a demyelinating neurological disease in an animal (for example a mammal such as a human), comprising administering to the animal: (a) a cell adhesion modulating agent as described herein that inhibits cadherin-mediated cell adhesion; and (b) one or more cells capable of replenishing an oligodendrocyte population; and thereby treating a demyelinating neurological disease in the animal.

Further provided is a method for facilitating migration of an N-cadherin expressing cell on astrocytes, comprising contacting an N-cadherin expressing cell with: (a) a cell adhesion modulating agent as described herein that inhibits cadherin-mediated cell adhesion; and (b) one or more astrocytes; and thereby facilitating migration of the N-cadherin expressing cell on the astrocytes.

The invention also provides a method for inhibiting synaptic stability in an animal (for example a mammal such as a human), comprising administering to the animal a cell adhesion modulating agent as described herein that inhibits cadherin-mediated cell adhesion, and thereby inhibiting synaptic stability in the animal.

Further provided is a method for modulating neurite outgrowth, comprising contacting a neuron with a modulating agent of the type herein described, and thereby modulating neurite outgrowth.

Also provided is a method for treating spinal cord injuries in an animal (for example a mammal such as a human) comprising administering to the animal a cell adhesion modulating agent as described herein that enhances neurite outgrowth, and thereby treating a spinal cord injury in the animal.

Additionally provided is a method for treating macular degeneration in an animal (for example a mammal such as a human), comprising administering to the animal a cell adhesion modulating agent as described herein that enhances classical cadherin-mediated cell adhesion, and thereby treating macular degeneration in the animal.

Also provided is a method for reducing unwanted cellular adhesion in an animal (for example a mammal such as a human), comprising administering to the animal with unwanted cellular adhesion a modulating agent as described herein and thereby reducing unwanted cellular adhesion, wherein the modulating agent inhibits cadherin mediated cell adhesion.

Further provided is a method for enhancing the delivery of a pharmaceutical active substance through the skin of an animal (for example a mammal such as a human), comprising contacting epithelial cells of the animal with a pharmaceutical active substance and a modulating agent as described herein and thereby enhancing the delivery of the substance through the skin, wherein the step of contacting is performed under conditions and for a time sufficient to allow passage of the substance across the epithelial cells, and wherein the modulating agent inhibits cadherin mediated cell adhesion.

Additionally provided is a method for inducing apoptosis in a cadherin-expressing cell, comprising contacting a cadherin-expressing cell with a modulating agent as described herein and thereby inducing apoptosis in the cell, wherein the modulating agent inhibits cadherin mediated cell adhesion.

Also provided is a method for preventing pregnancy in an animal (for example a mammal such as a human), comprising administering to the animal a modulating agent as described herein and thereby preventing pregnancy in the animal, wherein the modulating agent inhibits cadherin mediated cell adhesion.

Further provided is a method for facilitating blood sampling in a mammal, comprising contacting epithelial cells of a mammal with a cell adhesion modulating agent as described herein and thereby facilitating blood sampling in the mammal, wherein the step of contacting is performed under conditions and for a time sufficient to allow passage of one or more blood components across the epithelial cells, wherein the modulating agent inhibits cadherin mediated cell adhesion.

Additionally provided is a method for stimulating blood vessel regression, comprising administering to an animal (for example a mammal such as a human) a cell adhesion modulating agent as described herein and thereby stimulating blood vessel regression, wherein the modulating agent inhibits cadherin mediated cell adhesion.

The invention further provides a method for inhibiting endometriosis in a mammal, comprising administering to the mammal a cell adhesion modulating agent as described herein, wherein the modulating agent inhibits cadherin mediated cell adhesion.

Also provided is a method for enhancing inhaled compound delivery in an animal (for example a mammal such as a human), comprising contacting lung epithelial cells of the animal with a cell adhesion modulating agent as described herein and thereby enhancing inhaled compound delivery, wherein the modulating agent inhibits cadherin mediated cell adhesion.

Local disruption of cell-cell junctions in the skin or endothelial lining of blood vessels would increase permeability of the barrier and therefore improve access of drugs to the underlying tissues. E-cadherin, VE-cadherin and N-cadherin may be considered as principal targets here. The strategy would apply to topical application of drugs to the skin or oral mucosa or to anti-cancer drugs injected into tumour vasculature. The same logic applies to the blood-brain barrier (BBB) where control of permeability to drugs, especially in treating brain tumours, is a major problem. Cadherins make an important contribution to the BBB and its development. The current strategy used to increase permeability of the BBB, hyertonic shock, could be supplemented or replaced by a drug that disrupts cadherin-mediated adhesion via the salt bridge.

In addition to increasing vascular permeability for drug delivery to solid tumours, disruption of cadherin junctions in endothelial cells would also be expected to cause apoptosis. Not only do cadherin-cadherin interactions in endothelial cells maintain the integrity of the barrier, they also impart survival signals to endothelial cells. A cadherin antagonist injected locally into solid tumour vasculature would therefore disrupt the blood supply and cause tumour shrinkage.

A hallmark of malignant tumours is a propensity for invasiveness and metastatic spread. Adhesion by the principal epithelial cadherin, E-cadherin, is usually reduced in cancer and this is a consequence of mutational damage to the molecule or to decreased cell surface expression. Reduction in E-cadherin function is often accompanied by an increase in expression of N-cadherin which triggers signalling pathways which cause epithelial/mesenchymal transition and invasive behaviour. The present disclosure points the way to the rational design of drugs which would increase the affinity of E-cadherin-mediated cell adhesion by a large factor. An ideal strategy would be to combine this with an specific inhibitor of N-cadherin function. A drug to increase the affinity of E-cadherin-mediated adhesion may be designed to prevent intramolecular docking of Trp2 in domain 1 and formation of the intramolecular salt bridge, but would permit intermolecular βA strand exchange and formation of the salt bridge in trans. We have demonstrated the feasibility of this approach with our experimental cadherin constructs (see below). Our understanding of the strand exchange mechanism encourages optimism that drugs could be used to increase affinity of E-cadherin adhesion by several orders of magnitude. This effect is likely to counteract the deficiencies in cell adhesion which cause metastasis. The strategy could very useful in pre-cancerous conditions, particularly where topical application of the drug could be used to avoid possible adverse systemic effects. For example, in the mouth many oral cancers arise in pre-existing white patches (leukoplakia). In some cases these white patches are very widespread and management is a problem because it is not possible to remove them and it is also very difficult to determine whether they have become malignant. It would be feasible to apply a drug topically to these patches to increase epithelial adhesion and prevent invasion and cancer development. A similar strategy would be appropriate also for cervical cancer and for some skin cancers, e.g. basal cell carcinomas. These are specific examples but potential applications could be much wider, as would be appreciated by a person skilled in the art.

In another aspect, the invention may be used to prevent cancer (for example tumoural) cells from detaching and invading other tissue (anti-metastatic).

Pemphigus is a group of potentially life-threatening autoimmune diseases characterised by cutaneous and mucosal blistering. Pemphigus vulgaris is caused by failure in desmosomal cadherin-mediated adhesion due to the presence of autoantibodies against Dsg3 and/or Dsg1. Current treatment is centered on the use of immunosuppressive drugs and steroids to reduce antibody levels. A drug to enhance the affinity of the adhesive interaction between cadherins, both E-cadherin and/or desmosomal cadherins, would be very beneficial in maintaining integrity of the epidermal barrier. Similar logic applies to the treatment of other exfoliative skin diseases, e.g. Staphylococcal scalded skin syndrome and bullous impetigo.

### Biotechnological Applications of the Invention

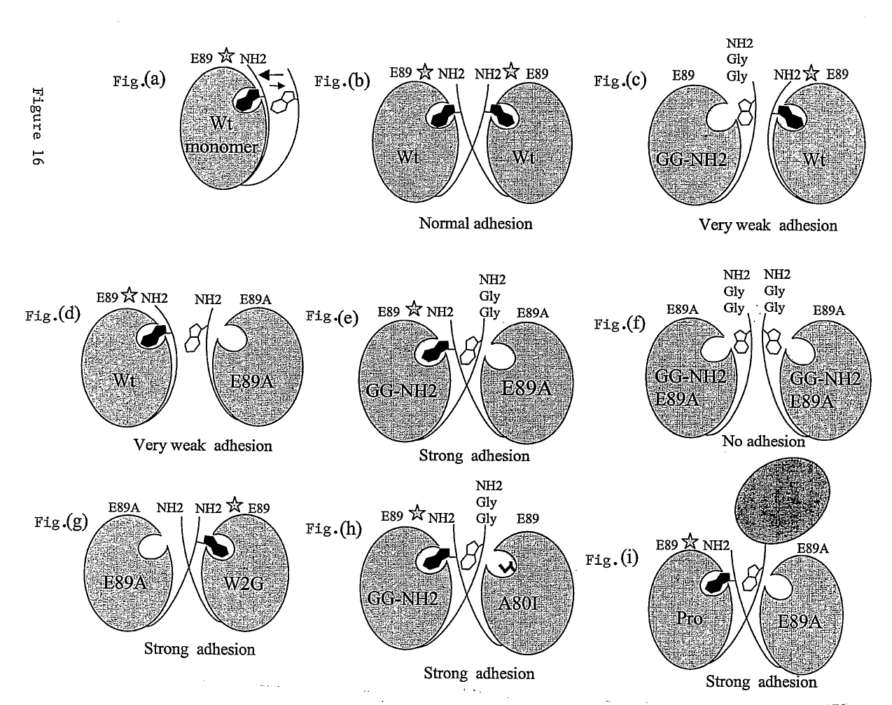

The mutant cadherins described herein that increase adhesion (for example E89A and the N-terminal extension Gly Gly; see also for example FIG. 16) act, in effect, like Velcro™. Each fails to stick to its own kind but will adhere strongly to the complementary molecule in a highly specific way. This property may be used in various laboratory procedures including cell selection applications, e.g. cell panning or magnetic bead methods. Mutant cadherin could be coated to a plastic plate or beads and cells to be positively selected would bear the opposing cadherin molecule attached via an antibody to a cell surface marker. The advantage of this system over conventional panning procedures (e.g. using antibodies coated to a plate) is that cadherin-mediated adhesion is Ca²⁺-dependent and can be quickly and completely disrupted simply by Ca²⁺ chelation. This contrasts with the harsh or less straightforward methods currently available with positive cell selection systems. The principle would be relevant to other lab procedures where specific adhesion and Ca²⁺-dependency offer advantages. The modified cadherin molecules, or active parts thereof, as described in the present invention may thus be used as a “reversible molecular glue”. With the present confirmation of the strand exchange mechanism we envisage that the affinity of the cadherin interaction could be increased by several orders of magnitude using point mutations. This would make the system a very useful adjunct to current laboratory methods for purifying cells and molecules.

In one biotechnological application, a first modified cadherin molecule according to the invention and a second modified cadherin molecule according to the invention are provided such that the molecules exhibit enhanced intermolecular adhesion. These first and second molecules are termed “SuperCadh-1” and “SuperCadh-2”, respectively, in the following two embodiments.

In the first embodiment, SuperCadh-1 and SuperCadh-2 may be used to isolate, quantify, identify and/or characterise one or more cells or molecules. SuperCadh-1 may be chemically linked to a binding substance such as streptavidin. This will provide a means to attach SuperCadh-1 into a SuperCadh-1-complex including an antibody adapted to bind with the binding substance, for example a biotinylated antibody which binds to SuperCadh-1 linked to streptavidin through the interaction of streptavidin with biotin. The antibody may be cross-reactive with the cell or molecule. SuperCadh-2 may be attached to a surface such as a plate (such as a plastic dish or well), matrix, bead or column. When placed into contact with each other in the presence of a medium containing calcium ions, SuperCadh-2 will adhere to SuperCadh-1 thereby allowing retention on the surface of the SuperCadh-1-complex including the cell or molecule. After optional washing steps, release of the SuperCadh-1-complex from the surface may be effected by removing calcium ions from the medium, for example using a chelating agent such as EGTA (ethylene glycol-bis[β-aminoethyl ether]-N,N,N′,N′-tetraacetic acid). In one example, CD4⁺ lymphocytes are isolated from whole blood using this method. Positively selected cells are recovered by adding EGTA and cell number and viability are then assessed.

In the second embodiment, a variation of the first embodiment above, there is provided a method for isolating, quantifying, identifying or characterising one or more cells which are genetically modified following insertion of a nucleotide sequence (for example, a gene or an RNAi molecule) of interest. Here, the one or more cells may be genetically modified also to express SuperCadh-1 on the surface of the one or more cells. In one example, the nucleotide sequence of interest and SuperCadh-1 may be co-expressed or expressed simultaneously by the cell (for example under the control of the same promoter). SuperCadh-1 expressed by the one or more cells may then be allowed to adhere to SuperCadh-2 in the presence of a medium containing calcium ions, thereby allowing retention of the one or more cells onto a surface such as a matrix. In this way, one or more cells which have been successfully genetically modified may be selected, isolated, characterised and/or purified. After optional washing steps, release of the cells expressing SuperCadh-1 from the surface may be effected by removing calcium ions from the medium, for example using a chelating agent such as EGTA. In one example, a vector encoding SuperCadh-1 and green fluorescent protein (GFP) is transfected into a variety of mammalian cells lines using standard techniques. Cell surface expression of SuperCadh-1 is used to select cells transfected with GFP by adhesion to a matrix coated with SuperCadh-2. Cell number and viability are assessed after release from the matrix by calcium removal using EGTA.

In each of the first and second embodiments described above, a simple, fast, non-aggressive and non-stressful method for cell or molecule recovery is provided. The simple addition of a chelating agent, for example, reverses adhesion between SuperCadh-1 and SuperCadh-2, allowing cell or molecule collection without the need for scraping, sorting by cytofluorometry, or addition of large quantities of peptides.

The invention also provides a method for reducing aggregation of cultured cells, comprising contacting cultured stem cells with a cell adhesion modulating agent as described herein and thereby reducing aggregation of stem cells, wherein the modulating agent inhibits cadherin mediated cell adhesion.

So that the manner in which the above-recited features, advantages and objects of the invention, as well as others which will become apparent, are attained and can be understood in detail, more particular description of the invention summarised above may be had by reference to the embodiments thereof illustrated in the accompanying drawings which form a part of this specification. It is to be noted, however, that the accompanying drawings illustrate only specific embodiments of the invention and are therefore not to be considered limiting of its scope as the invention may admit to other equally effective embodiments.

In the drawings:

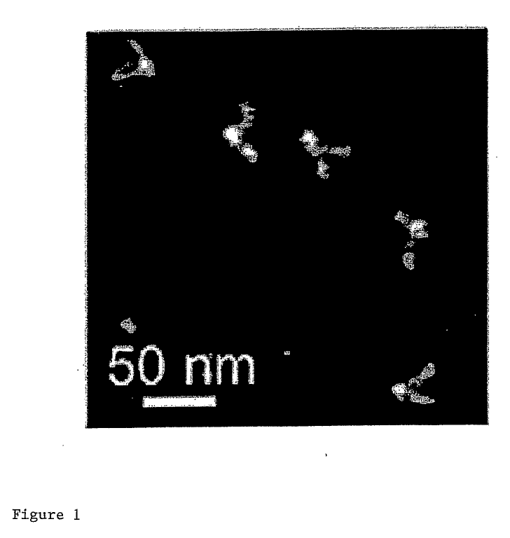

FIG. 1 is an atomic force image of a purified dimeric N-cadherin-Fc fusion protein adsorbed to a mica surface.

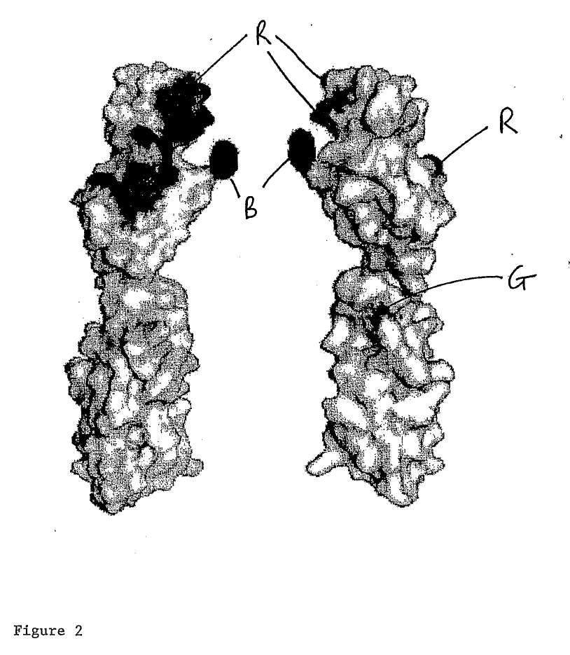

FIG. 2 (prior art) shows the two domain structure of N-cadherin, PDB 1NCJ (Tamura et al., 1998).

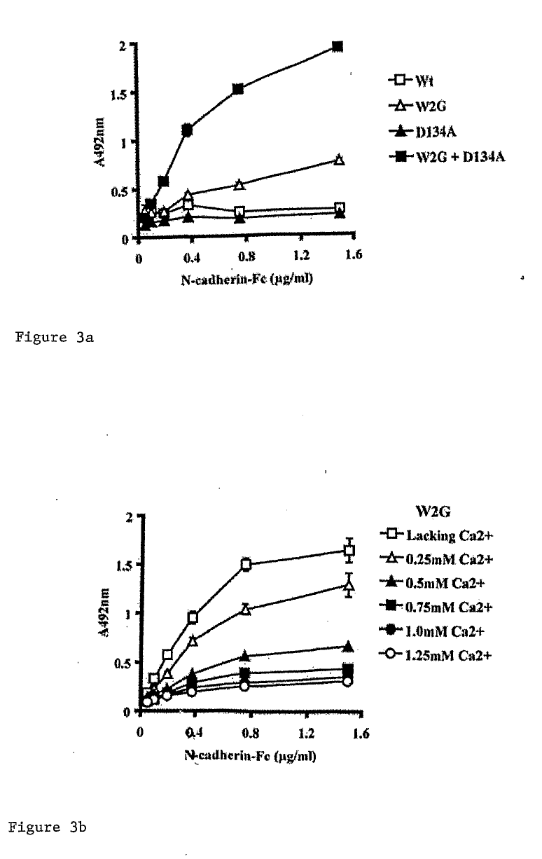

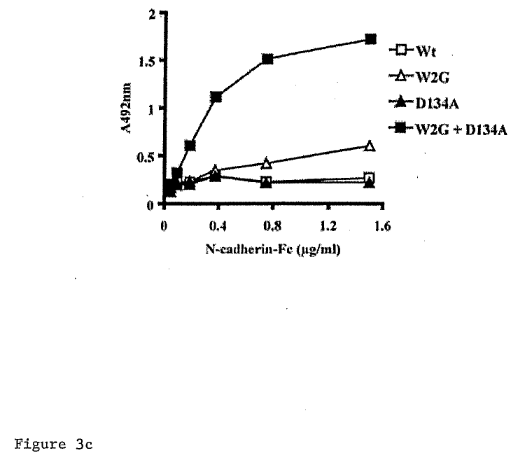

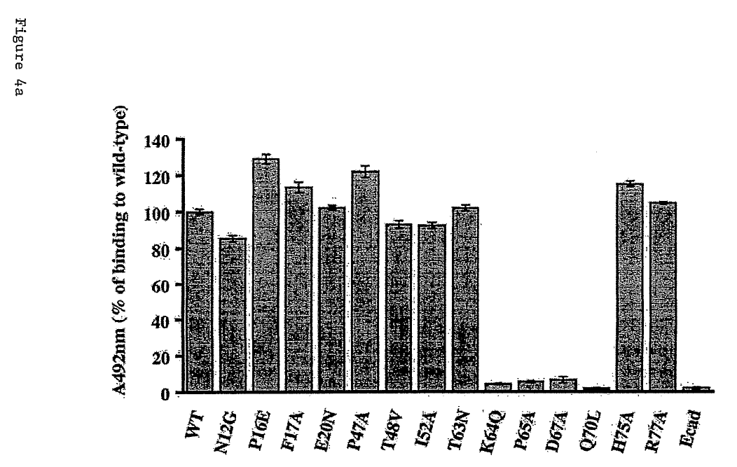

FIGS. 3(a)-(c)—are graphs showing the binding of peptide-specific antibody K7 to wild type and mutant N-cadherin-Fc fusion protein.

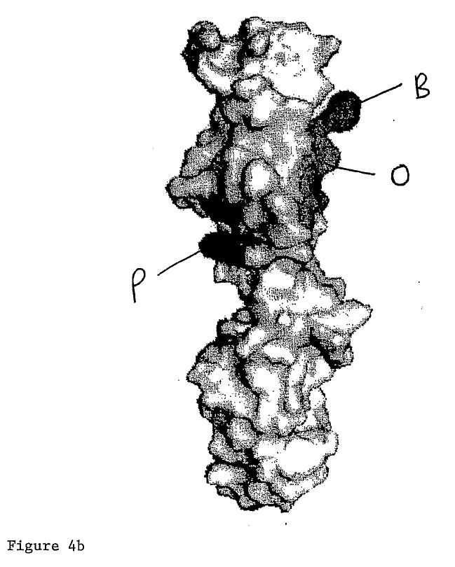

FIG. 4 shows the results of epitope mapping of antibody GC4.

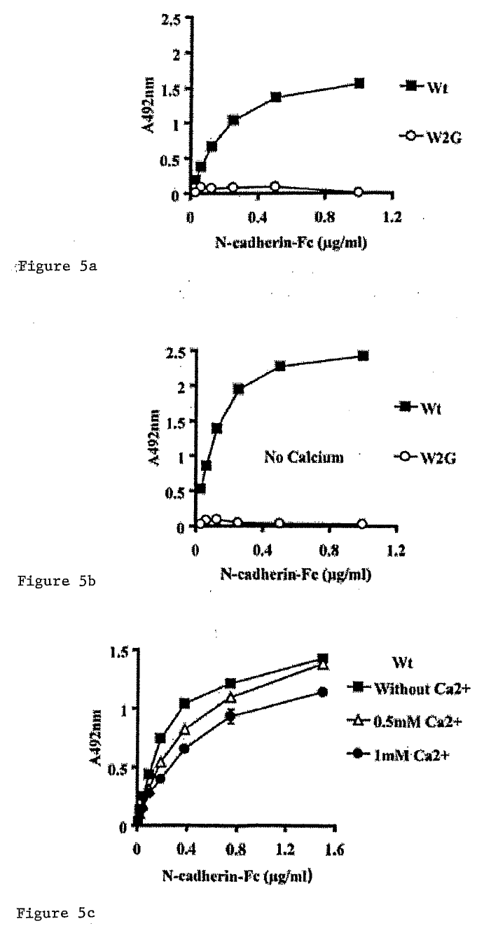

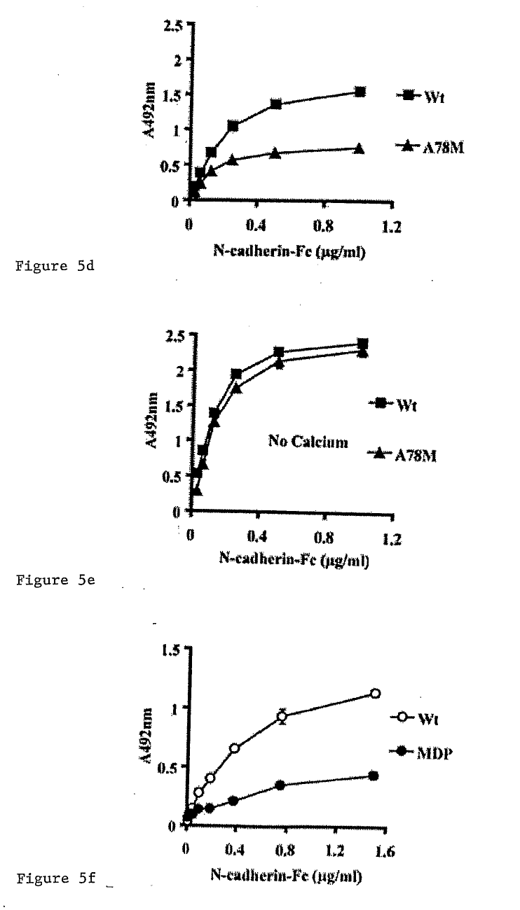

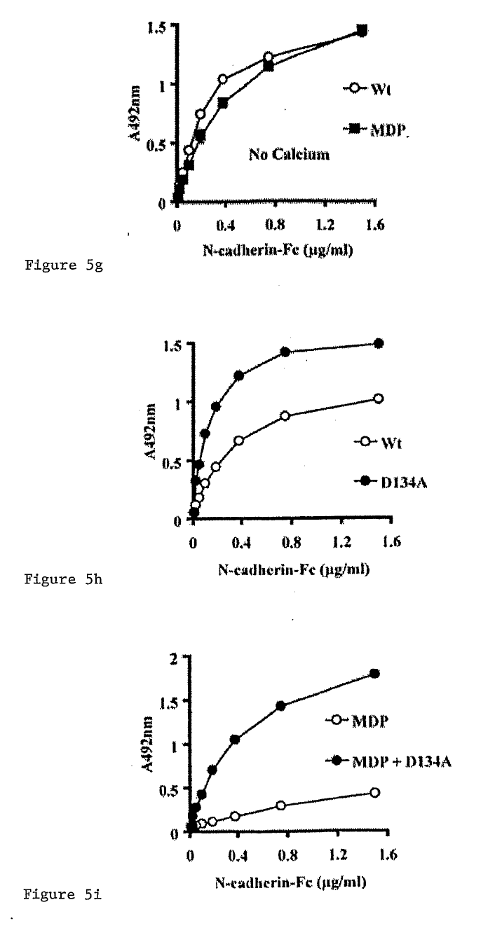

FIGS. 5(a)-(i) are graphs showing binding of mAb GC4 to dimeric N-cadherin-Fc mutants in the presence or absence of calcium.

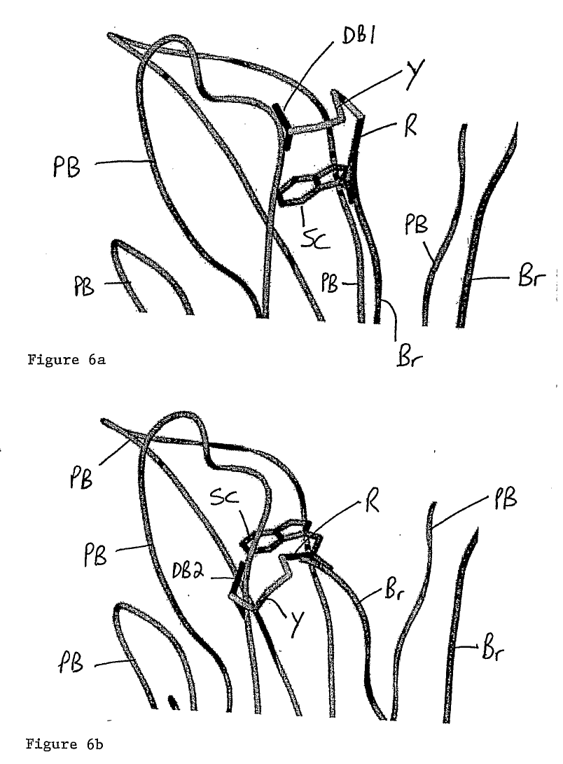

FIG. 6 models the formation of ‘reporter’ disulphide bonds formed during strand exchange in domain 1.

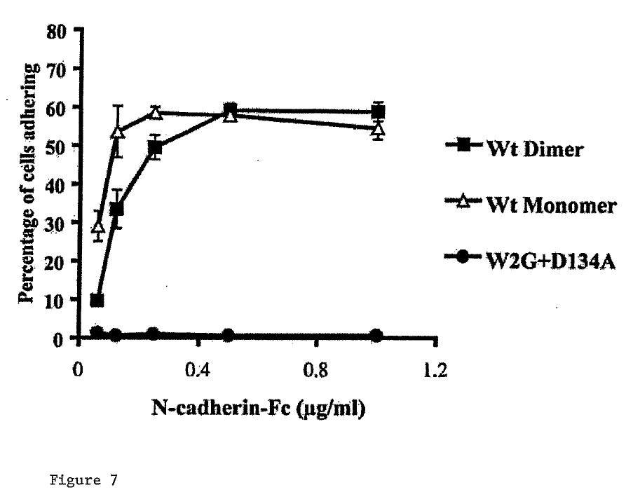

FIG. 7 is a graph showing a comparison of monomeric and dimeric N-cadherin-Fc in supporting adhesion of DX3 melanoma cells.

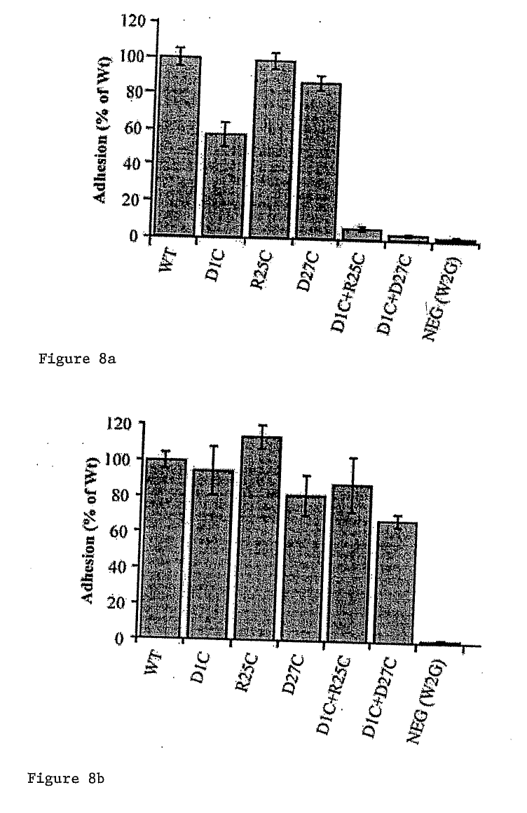

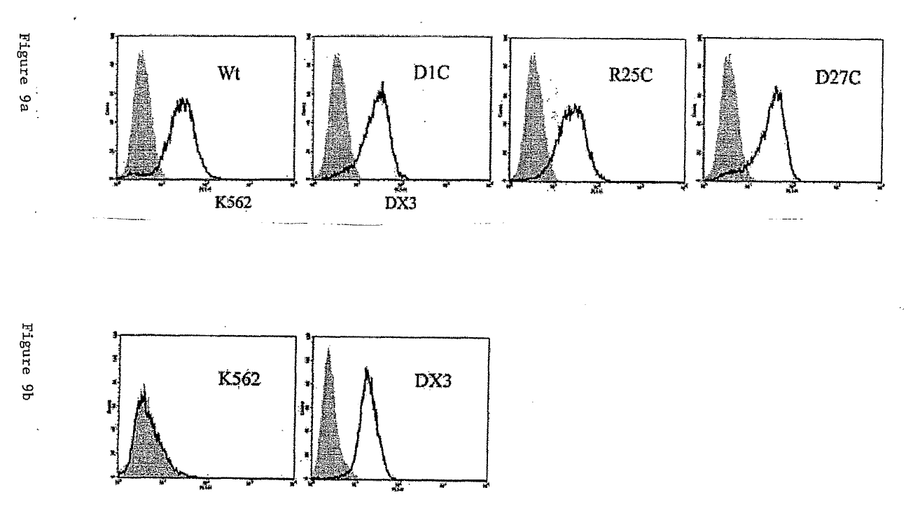

FIGS. 8(a) & (b) are graphs showing the effect of cysteine point mutations on the adhesive capacity of monomeric N-cadherin-Fc.

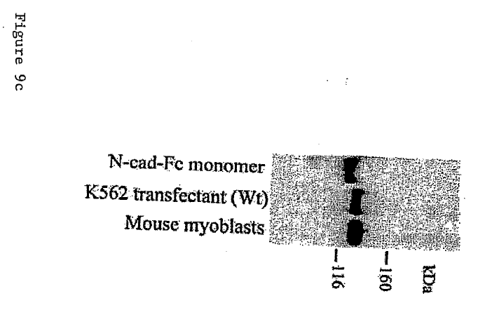

FIGS. 9(a)-(c) show expression of cell surface N-cadherin by K562 transfectants and by DX3 melanoma cells.

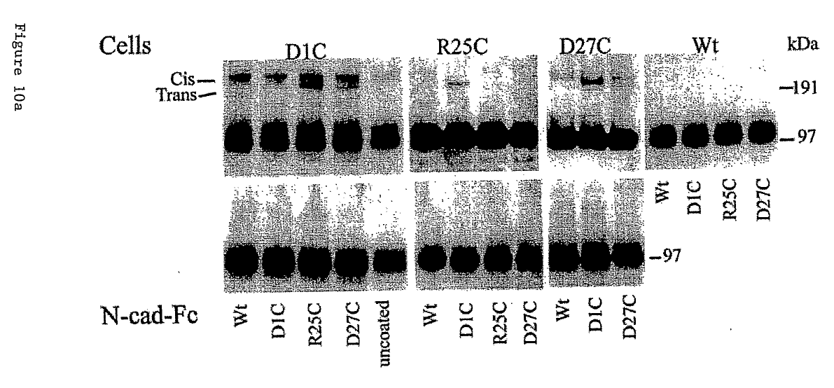

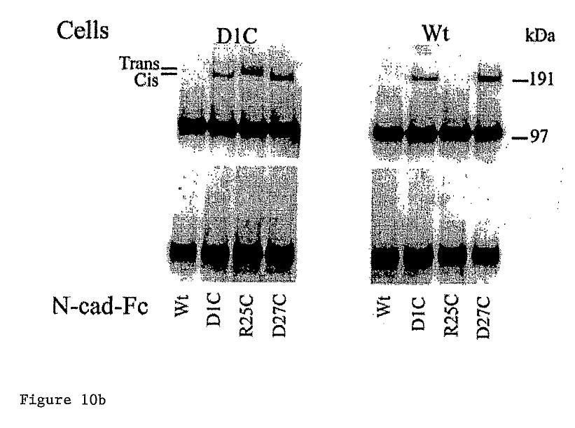

FIGS. 10(a) & (b) are Western blots showing the formation of ‘reporter’ disulphide bonds during cadherin-mediated cell adhesion.

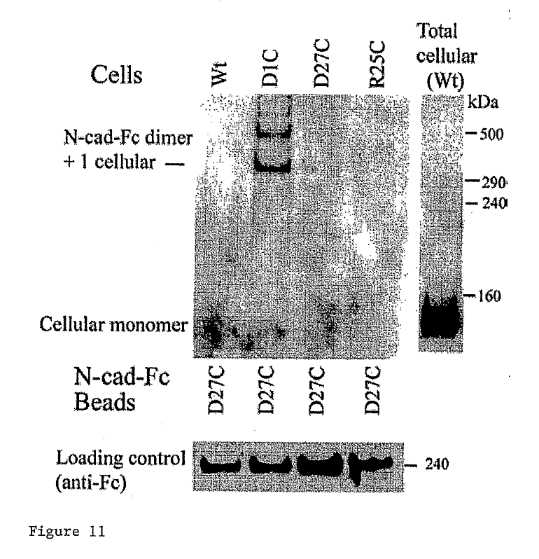

FIG. 11 shows the isolation of disulphide-bonded trans-dimers.

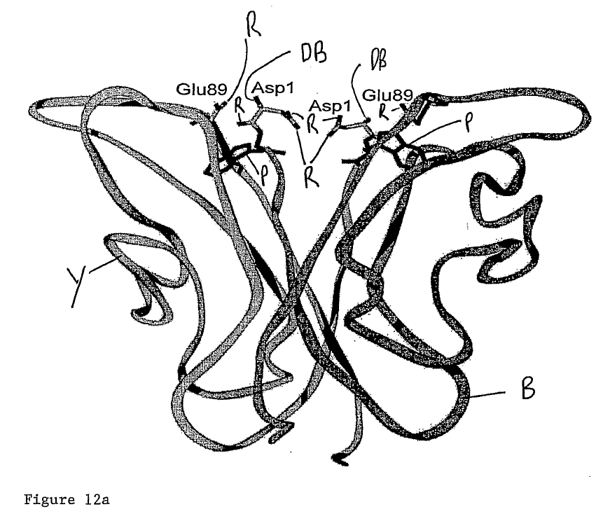

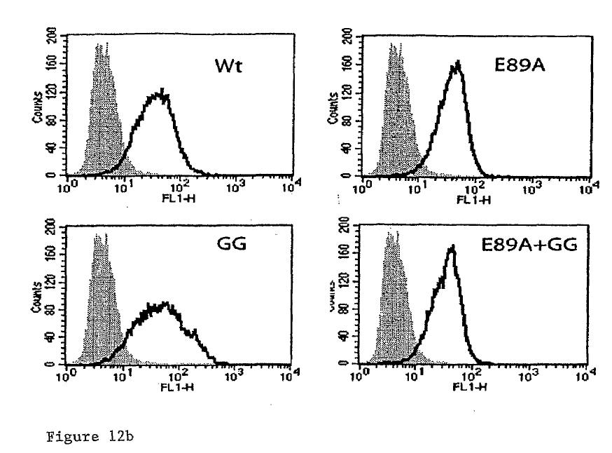

FIG. 12(a) is a graphic and (b) shows immunofluorescent staining results, pertaining to the salt bridge between Asp1 and Glu 89 of N-cadherin.

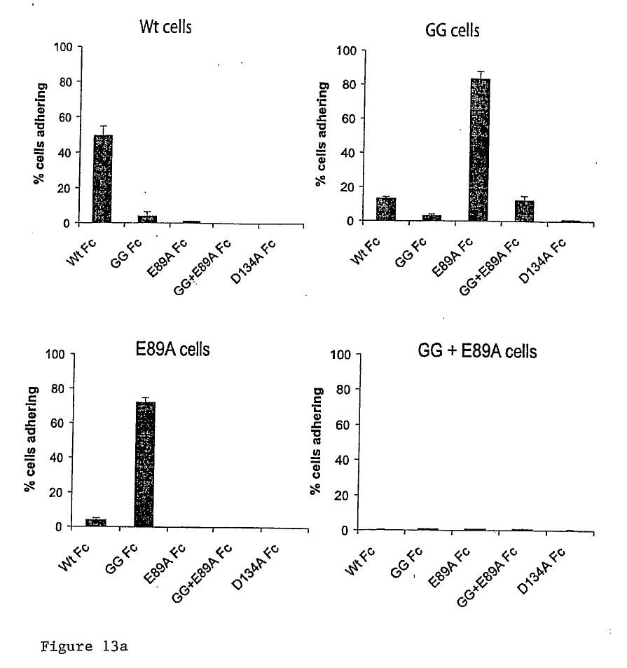

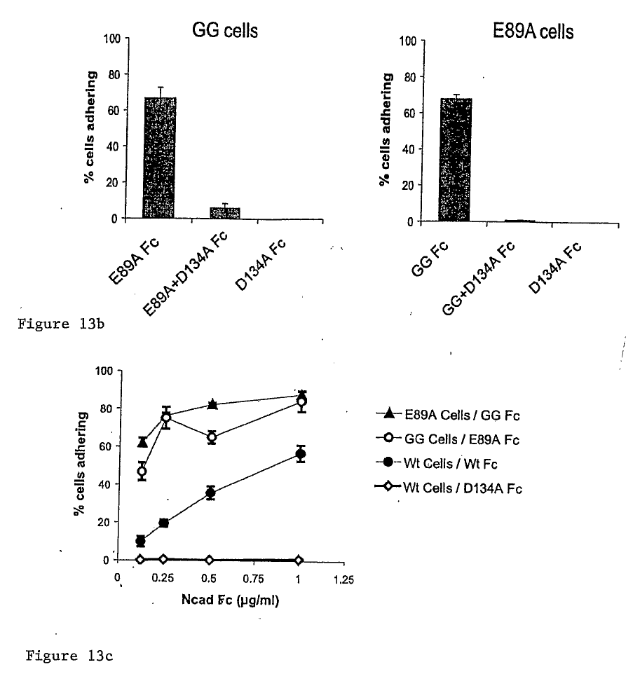

FIG. 13(a)-(c) are graphs showing the effect of the salt bridge on cadherin-mediated cell adhesion.

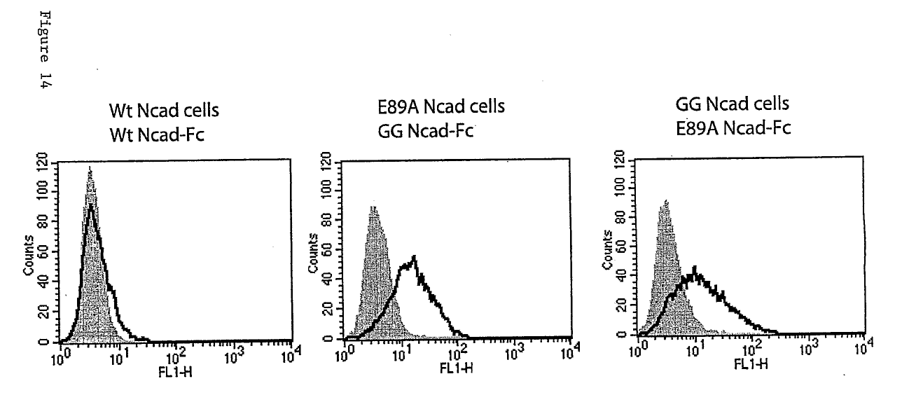

FIG. 14 provides immunofluorescent staining results showing binding of soluble N-cadherin Fc to cell surface N-cadherin.

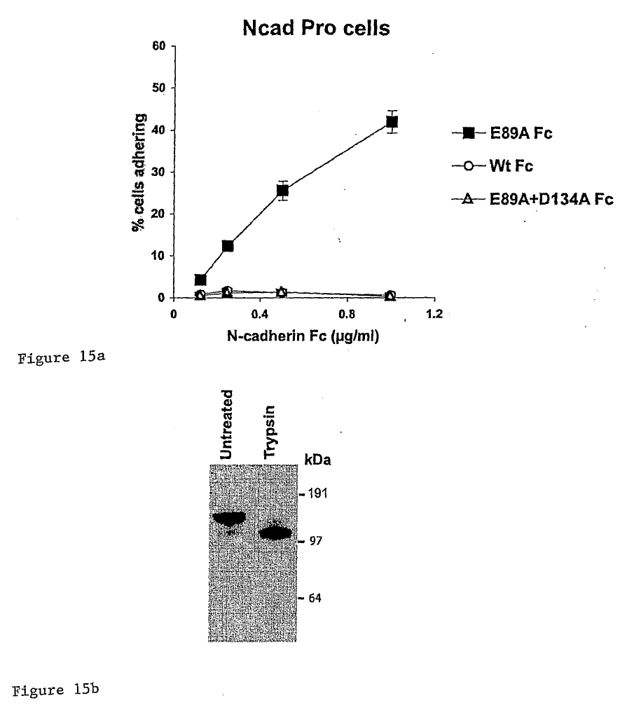

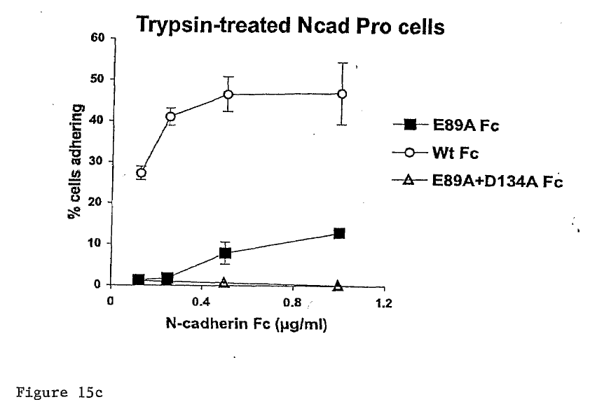

FIG. 15(a), (c) are graphs and (b) is a western blot, relating to effect of the N-cadherin prodomain on adhesion.

FIG. 16 is a graphic showing the adhesion in different combinations of cadherin mutants and in wild-type situation.

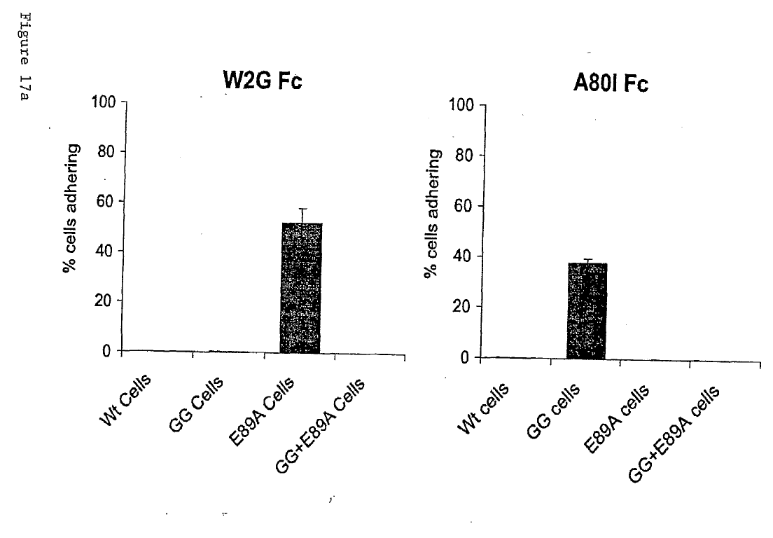

FIG. 17(a) shows graphs and (b) shows photographs, demonstrating the effect on binding of removing Trp2 or blocking the hydrophobic acceptor pocket of N-cadherin.

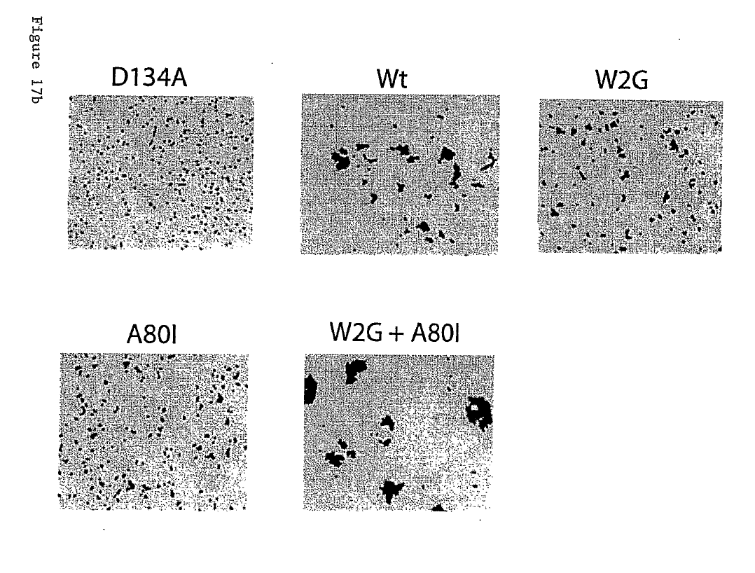

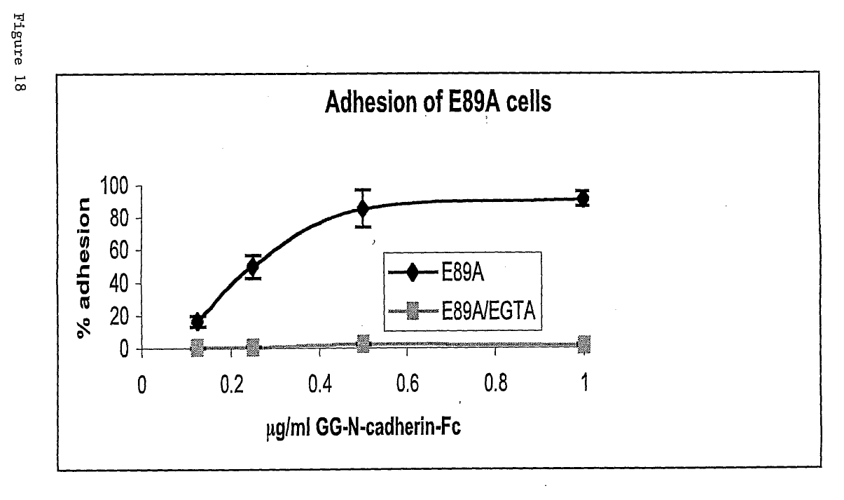

FIG. 18 is a graph showing the reversal of adhesion between N-cadherin mutants by chelation of calcium.

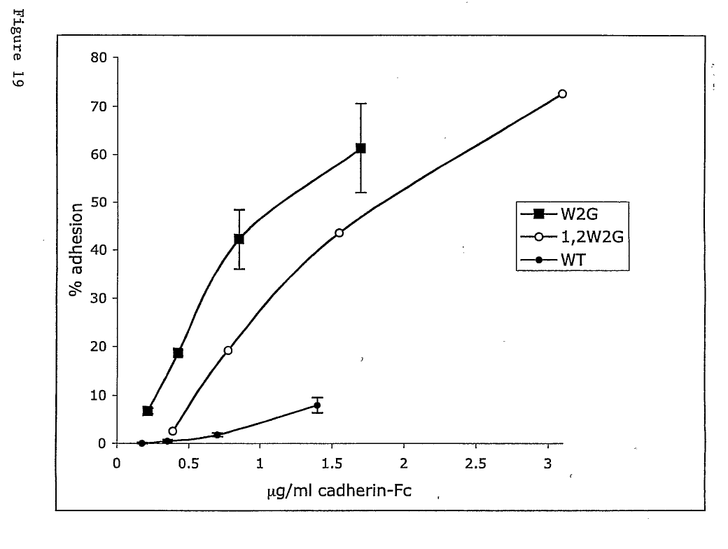

FIG. 19 is a graph relating to the adhesion of a fragment of N-Cadherin mutant.

The figure legends in more detail are as follows:

In FIG. 1, the two curved ‘arms’ in each molecule are the five extracellular domains of N-cadherin which are seen to be joined to the Fc region. The thickness of the deposited molecules is reflected in their shading and approximate values were obtained; 5 nm for the more intensely white Fc region and 3 nm for the cadherin domains.

In FIG. 2, the location of the 13-mer peptide in domain 1, against which antiserum K7 was prepared, is labelled “R” (red). Trp2 is not integrated into the domain fold and is labelled “B” (blue). The position of the D134A mutation is labelled “G” (green). The two images show opposite faces of domain 1.

In FIG. 3(a), the mutations W2G and D134A in N-cadherin Fc, dimerised via Fc, were tested singly or in combination and compared with results from the wild type molecule. Calcium was present in the assay at 1.25 mM. In FIG. 3(b) N-cadherin-Fc mutant W2G was pre-equilibrated with varying levels of Ca2+ which were maintained throughout the assay. In FIG. 3(c) monomeric N-cadherin-Fc was tested for comparison with the Fc-dimerised form; 1.25 mM Ca2+ was present.

FIG. 4(a) shows binding of mAb GC4 to dimeric N-cadherin Fc bearing point mutations, in the presence of 1.25 mM calcium. In FIG. 4(b), amino acids K64, P65, D67 and Q70 are labelled “P” (purple) and are shown relative to the position of nonintercalated Trp2 (labelled “B” [blue]) and the #A strand (labelled “0” [orange]).

In FIG. 5(a), wild type (Wt) and the W2G mutant are compared in the presence of 1.25 mM Ca2+. In FIG. 5(b) the same titration was performed in the absence of Ca2+; the two titrations were performed together in the same assay plate and values can be compared directly. Results in the presence of EGTA (not shown) were almost identical to those in FIG. 5(b). In FIG. 5(c), wild type N-cadherin-Fc was tested at varying levels of calcium. In FIG. 5(d) the hydrophobic pocket mutant A78M was compared with wild type N-cadherin in the presence of 1 mM calcium. In FIG. 5(e) the comparison was made in the absence of calcium. Similarly, a comparison between the N-terminal extension mutant MDP and wild type N-cadherin was made in the presence [FIG. 5(f)] or absence [FIG. 5(g)] of 1 mM calcium. Finally, coordination of calcium in the domain 1-2 junction was disrupted using the mutation D134A, while retaining calcium (1 mM) in the assay buffer. The effect of this mutation, compared with wild type, is shown in FIG. 5(h). The greater effect of D134A on the MDP extension mutant is shown in FIG. 5(i).

In FIG. 6, structures are derived from PDB 1NCI (Shapiro et al., 1995). The two opposing N-cadherin domains are labelled “PB” (pale blue) and “Br” (brown), respectively. The side chain of Trp2 (labelled “SC”) is shown located in the hydrophobic pocket of the “PB” domain. In (a) the backbone of C1 is labelled “R” (red) while the side chain (“SC”) forms a disulphide bond 26 (labelled “Y” [yellow]) with C27 (labelled “DB1”). In (b) the disulphide bond (“Y”) is formed between C1 (“R”) and C25 (labelled “DB2”).

In FIG. 7, cadherin preparations, shown to be monodisperse, were titrated onto an assay plate coated with goat anti-human Fc. DX3 Cells were applied and adhesion was assessed as described. Dimeric N-cadherin-Fc containing the mutations W2G and D143A provided a negative control.

In FIG. 8(a), the assay was conducted in HBSS+2% FCS (oxidising conditions). In FIG. 8(b), reducing conditions were established by adding 10 mM DTT. Approximately 65% of wild type cells adhered to the plate in both assays. Reduction restored adhesive capacity of the double mutants D1C,R25C and D1C,D27C, respectively.

In FIG. 9(a), stable K562 transfectants were stained with mAb NCD-2 to chicken N-cadherin. The four panels show comparable levels of expression. In FIG. 9(b), untransfected K562 Cells were tested with mAb 8C11 to human N-cadherin and the first panel verifies that these cells do not naturally express N-cadherin. The final panel shows DX3 Cells stained with 8C11, showing strong expression of human N-cadherin. In FIG. 9(c), the molecular size of cellular N-cadherin from K562 transfectants (Wt) is compared with that of monomeric N-cadherin Fc fusion protein (Wt) bearing the mutations F405A,Y407A in the Fc region to prevent dimerisation. N-cadherin extracted from normal myoblast cells is also shown for comparison. The gel was run under non-reducing conditions and blotting was conducted using a mixture of pan cadherin antibody and anti-Fc.

In FIG. 10, K562 Cells expressing wild type or mutant N-cadherin were allowed to adhere, under reducing conditions, to a panel of monomeric N-cadherin-Fc molecules bearing the same set of mutations. Oxidising conditions were then restored and the formation of disulphide bonds was assessed by SDS-PAGE and immunoblotting as described. In FIG. 10(a) the blot was developed with antibody to cellular cadherin cytoplasmic domain. The upper panels show gels run under non-reducing conditions. Disulphide-bonded trans-dimers can be seen to form only when cadherin molecules bearing complementary cysteine point mutations were apposed. In contrast, the D1C and D27C mutations allowed formation of disulphide-bonded cis-dimers on the cell surface. With wild type cells (right panel) no disulphide-bonded species are seen. The track labelled ‘uncoated’ in this series reflects a small degree of background adhesion to wells lacking cadherin and shows the position of the cis-dimer. The lower panel in FIG. 10(a) shows the same preparations run under reducing conditions. In FIG. 10(b) a similar experiment was conducted, the blot being developed with anti-Fc. Again the trans-dimer can be seen when mutations D1C and R25C were apposed. As in FIG. 10(a), cis-dimers were formed by D1C and D27C mutants but not by R25C. Cisdimers formed by monomeric N-cadherin-Fc are seen to run slightly below the transdimer in contrast to the situation in FIG. 10(a) where the cellular cis-dimer runs above the trans species.

In FIG. 11, magnetic beads coated with N-cadherin-Fc bearing the mutation D27C were allowed to stick to K562 Cells expressing wild type N-cadherin or the mutants D1C, R25C or D27C. After formation of disulphide bonds, the trans-species attached to the beads were isolated from cis-dimers and non-involved cell surface N-cadherin. The trans-dimers were detected by immunoblotting for cellular N-cadherin cytoplasmic domain. The upper panel shows that trans-dimers formed only with the combination D27C beads adhering to D1C cells. The main band at about 300 kDa represents N-cadherin-Fc (dimerised by the Fc hinge region) disulphide-bonded to one cellular cadherin molecule. Higher order assemblies are also seen. The right hand panel shows total cellular cadherin for comparison. The lower panel shows a gel loading control.

FIG. 12 (a) shows strand exchange in the C-cadherin structure 1L3W. The two domain 1 protomers labelled “B” (blue) and “Y” (yellow), respectively, and Trp 2 is labelled “P” (purple). Juxtaposition of the nitrogen of the N-terminal amino group of Asp 1 (“DB”—dark blue) and the oxygen atoms of the acidic side chain of Glu 89 (“R”—red) are shown. Panel (b) shows cell surface staining of wild type and mutant N-cadherins expressed by K562 transfectants, matched for equal expression. The mutations E89A and GG (in which the N-terminus was extended by two glycine residues) were present singly or together in the same molecule. The shaded profile is a negative control using FITC-labelled second antibody only.

FIG. 13(a) shows adhesion of N-cadherin transfectants to wild type or mutant N-cadherin Fc fusion proteins coated at 1 g/ml. Extension of the N-terminus by two amino acids, GG, or the mutation E89A inhibited adhesion to wild type N-cadherin but, when present in opposing molecules, they formed a complementary pair resulting in enhanced adhesion. The mutation D134A prevents co-ordination of calcium in the junction between domains 1 and 2 and served as a negative control. FIG. 13(b) shows that adhesion between E89A and, the GG extension mutant was ablated by the mutation D134A. FIG. 13(c) illustrates enhanced adhesion between the complementary pair, GG and E89A, compared with adhesion between wild N-cadherin type molecules over a range of concentrations of the Fc fusion proteins.

In FIG. 14, mutant or wild type Fc fusion proteins were used to ‘stain’ cell surface N-cadherin expressed by K562 transfectants. Binding was detected with FITC-labelled anti-Fc. The shaded profiles are negative controls using N-cadherin Fc with the mutation D134A. Results show that the affinity between wild type cadherin molecules was too low to give detectable binding, whereas the interaction between E89A and the GG mutant gave strong staining.

In FIG. 15, L cells expressing N-cadherin with an uncleaved prodomain were tested for adhesion to mutant or wild type N-cadherin Fc fusion proteins. FIG. 15(a) shows that the L cells adhered to the E89A mutant but not to wild type N-cadherin; in FIG. 15 (b), a western blot demonstrates removal of the prodomain by treatment of the L cell transfectants with trypsin; in FIG. 15(c) after treatment, the L cells adhered strongly to wild type N-cadherin whereas adhesion to the E89A mutant was greatly diminished.

A full explanation of FIG. 16 is provided below. Briefly, FIG. 16(a) shows domain 1 in isolation. An equilibrium between docked and undocked Trp2 favours the docked form because the salt bridge (shown as a star) between E89 and the N-terminus stabilises Trp2 insertion. FIG. 16(b) shows adhesion between wild type molecules. Adhesion is moderate. In FIG. 16(c) the salt bridge on one side is prevented by extension of the N-terminus and in FIG. 16(d) by the mutation E89A. In both situations adhesion is very weak. Although one strand can cross-intercalate, the process competes unfavourably with intramolecular docking of Trp2 into the wild type domain. In FIG. 16(e) the two mutations form a complementary pair. Intramolecular docking is prevented and therefore the activation barrier for strand exchange is lowered. Exchange of one strand is possible and cross-intercalation of Trp2 is stabilised by the salt bridge. Adhesion is enhanced. In FIG. 16(f) the double mutation is present on each side. The salt bridge cannot form to support strand exchange so there is no adhesion.

In FIG. 16(g)-(i), binding of further complementary cadherin mutants providing enhanced adhesion is shown. In FIG. 16, a transition state in which Trp2 is undocked is sampled from either side and is depicted as an ‘unshaded’ tryptophan.

In FIG. 17(a) K562 Cells expressing wild type or mutant N-cadherin were tested for adhesion to N-cadherin Fc (1 μg/ml) bearing either the mutation W2G or the pocket-blocking mutation A80I. The W2G mutant acted as a strand acceptor and therefore adhered to cells expressing the E89A mutation. In contrast, the A80I mutant behaved as a strand donor because intramolecular docking of Trp2 was denied and therefore bound to cells expressing the GG N-terminal extension mutant. In FIG. 17(b) dynabeads coated with the W2G mutant or the A80I mutant were tested for aggregation separately or as a mixture and compared with aggregation mediated by wild type N-cadherin. The D134A mutant served as a negative control.

In FIG. 18, K562 Cells expressing the N-cadherin mutation E89A were allowed to adhere for 30 minutes at 37 C to N-cadherin-Fc having the GG extension mutation, immobilised to an assay plate. Adherent cells were then released by washing the plate with 2 mM EGTA and residual adherent cells were quantitated. The results show that calcium chelation with EGTA released almost all adherent cells.

Finally, in FIG. 19 K562 Cells expressing N-cadherin with the mutation E89A were allowed to adhere to N-cadherin Fc, coated to an assay plate, having the mutation W2G. The W2G mutant was tested either as a full length construct (domains EC1-EC5) or as a truncated construct (identified on the graph as 1,2 W2G) having only domains EC1 and EC2. The truncated construct was only slightly less efficient in supporting adhesion than the full length version.

### EXPERIMENTAL

### 1. Example 1

### The Mechanism of Cell Adhesion by Classical Cadherins: the Role of Domain 1

In the present example we have used antibodies to detect conformational changes in EC1 of N-cadherin, prepared as an Fc-fusion protein, to investigate the effect of Trp2 and Ca²⁺ on the stability of this domain. In addition we have investigated the effect of calcium on the propensity of Trp2 to dock into a hydrophobic pocket in its own domain. Finally, we provide persuasive evidence for strand exchange as the primary event in adhesion. A novel strategy has been used involving the formation of a ‘reporter’ disulphide bond which captures mutant cadherin molecules in trans-alignment as cells undergo adhesion. This bond can form only if the molecules are orientated by strand exchange.

### 1.1 Materials and Methods

### Antibodies to N-Cadherin

A polyclonal sheep antiserum was prepared by standard methods against the synthetic peptide PQELVRIRSDRDK SEQ ID No 2, which spans the MB strand of chicken N-cadherin. The peptide was conjugated to keyhole limpet haemocyanin for immunization. Preliminary experiments established that this antibody did not react with wild type N-cadherin-Fc in its native conformation but gave a strongly positive result with N-cadherin-Fc that had been partially denatured by direct adsorption to a plastic surface. The rat mAb NCD-2, specific for an epitope in the BC loop of domain 1 of chicken N-cadherin, was obtained from R & D Systems (code BTA6). Mouse mAb 8C11, specific for domain 4 of human N-cadherin, was a gift from Dr M J Wheelock (Puch et al., 2001) and mouse mAb GC4 (also known as GB-9) was obtained from Sigma (code C2542). A rabbit pan anticadherin antiserum specific for a conserved sequence of 24 amino acids in the cytoplasmic domain (Sigma, code C3678) was used for immunoblotting.

### Antibody Binding Tests

Antibody binding to N-cadherin-Fc fusion proteins was detected by enzyme-linked immunosorbent assay (ELISA) based on a previously described method (Corps et al., 2001). Briefly, assay plates were coated with varying levels of monomeric or dimeric Ncadherin Fc-fusion proteins via rabbit or goat anti-human Fc. Assay plates were then pre-equilibrated for 7 minutes with varying levels of calcium chloride added to calcium-free Hanks balanced salt solution (HBSS), containing 0.075% Tween 20. Antiserum K7 (1:75) or mAb GC4 (2 μg/ml) was then added in IBSS containing the appropriate level of calcium and incubated for 1 hour at room temperature. Antibody binding was detected with anti-sheep or anti-mouse HRP-labelled secondary antibody. Assays were conducted in duplicate or triplicate and results are presented as mean +/−sem.

### Design of Cysteine Point Mutations

Predictions of disulphide bond formation were based on the strand exchange structure PDB 1NCI (Shapiro et al., 1995). It was viewed and manipulated using Swiss PDB Viewer (http://www.expasy.org/spdbv/). Two extra amino acids at the N-terminus of this structure were removed to give the correct sequence. Alternative pairs of mutations, D1C,R25C or D1C,D27C, were introduced so that either pair would form a disulphide bond between the two domain 1 protomers; numbering refers to the mature cadherin protein. Formation of the C1-C25 disulphide bond required torsion, within Ramachandran limits, of psi angles in the α-carbon backbone of the βA strand in the vicinity of Val3, while maintaining the side chain of Trp2 in an unchanged position. Formation of the C1-C27 disulphide bond required only rotation of the side chain of C1.

Adjustments were also made to the side chains of C25 and C27. After energy minimisation, the beta carbon atoms of the paired cysteines for both bonds were within 4A, which is optimal for disulphide bond formation. There were no amino acid clashes in either case.

### DNA Constructions and Transfections

Full length chicken N-cadherin cDNA in pcDNA3.1 was obtained from Prof. P Doherty, King's College, London. The point mutations D1C, R25C and D27C were introduced using a QuikChange mutagenesis kit (Stratagene). Mutant and wild type constructs were stably transfected into the human myeloid leukemic cell line K562 by electroporation and selection in G418 (1 mg/ml G418 in DMEM+10% FCS). Clonal cell lines were obtained by limiting dilution and were matched for equal expression of N-cadherin by cell surface immunofluorescent staining and flow cytometry. Chicken N-cadherin-Fc fusion protein linked by disulphide bonds at the Ig hinge region to form a dimer was prepared as follows: cDNA for the five extracellular domains of N-cadherin, coding up to the amino acid sequence GLGT, was isolated by PCR and cloned into the vector pIgSig (R&D Systems). This vector provides a signal sequence from CD33 and adds the CH2 and CH3 domains and the hinge region of human IgG1 heavy chain. The construct was modified to produce monomeric N-cadherin-Fc by introducing the mutations F405A and Y407A into the CH3 domain of Fc (Dall'Acqua et al., 1998) which prevent dimerisation of this domain. The fidelity of all DNA constructs was verified by sequencing.

Soluble N-cadherin-Fc fusion protein was obtained by transient transfection of Cos7 Cells as previously described (Corps et al., 2003). Wild type monomeric N-cadherin-Fc and the double mutant W2G,D134A were checked for molecular size by gel filtration on a Superdex-200 PC3.2/30 Column equilibrated with 50 mM Tris.Cl, pH 7.4, 150 mM NaCl, 1 mM CaCl2 and were shown to be monodisperse and monomeric. Soluble N-cadherin-Fc was routinely quantitated using an ELISA for Fc, standardised against purified cadherin-Fc fusion proteins.

### Cadherin-Mediated Cell Adhesion Tests

96 well immunoassay plates (Costar) were coated overnight with affinity-purified goat anti-human Fc (Sigma, code 12136) at 5 μg/ml in PBS, then blocked with 1% BSA for 2 hours at room temperature. Monomeric or dimeric N-cadherin-Fc fusion protein in Cos cell supernatants was added as described for the ELISA assay. DX3 Cells, a human melanoma cell line which expresses N-cadherin, were dissociated with Cell Dissociation Solution (Sigma, code C5789), resuspended in HBSS with 2% FCS and assessed for adhesion to wild type or mutant N-cadherin-Fc as previously described (Corps et al., 2001). Microscopic examination established that the cells were present as a single cell suspension as they settled onto the plate. For assays conducted in reducing conditions, 10 mM DTT was present during the adhesion and washing steps. Determinations were conducted in triplicate and results are presented as mean +/−sem.

### Formation of Reporter Disulphide Bonds During Cell Adhesion

Monomeric N-cadherin-Fc (1 μg/ml) bearing the three alternative cysteine point mutations, D1C, R25C or D27C, was immobilised on 96-well plates with goat antihuman IgG Fe as previously described for E-cadherin-Fc (Corps et al., 2001). Unbound cadherin was removed by washing with HBSS, 0.1% BSA, followed by HBSS alone. The plates were not blocked with additional protein. K562 transfectants expressing N-cadherin bearing complementary or non-complementary point mutations (6×104 Cells in 100 μl HBSS containing 10 mM DTT), in single cell suspension, were added to the coated wells which contained 100 μl HBSS. The final concentration of DTT was therefore 5 mM DTT during the adhesion stage. The cells were allowed to settle for 10 min at room temperature before incubation at 37° C. for 30 minutes to complete adhesion. Microscopic examination of the wells before washing showed that the cells were not clumped but remained as a carpet of single cells on the surface of the plate. Non-adherent cells were then removed by washing with HBSS without DTT to restore oxidising conditions (4-6 washes over 15 to 20 minutes). Adherent cells from a pool of 4 wells for each experimental condition were then solubilised in sample buffer for SDS-PAGE and analysed on NuPAGE Novex gradient gels, 3-8% or 4-12%, (Invitrogen), under nonreducing or reducing conditions. Cellular cadherin was detected by immunoblotting using rabbit pan anti-cadherin antiserum specific for the cytoplasmic domain. Alternatively, N-cadherin-Fc fusion protein was detected with rabbit anti-human IgG, Fc-specific (Pierce, code 31142). The secondary antibody for both was peroxidase-conjugated AffinPure goat anti-rabbit IgG, F(ab′)2 fragment-specific (Jackson ImmunoResearch Labs, code 111-035-006).

In an alternative protocol, Dynabeads (Dynal) coupled to Protein A were coated with dimeric N-cadherin-Fc (1 μg/ml), bearing the mutation D27C, for 1 hour at room temperature in the presence of 0.1% Tween 20 and 4 mM EGTA (to prevent aggregation). The beads were then washed with HBSS. K562 transfectants expressing cell surface Ncadherin with the mutations D1C, R25C or D27C, were treated with 10 mM DTT for 15 minutes at 37° C., then washed and resuspended in HBSS without DTT. Cells (2.4×105) were mixed with 3 μl of beads coated with mutant N-cadherin-Fc in a final volume of 100 μl HBSS. Cells and beads were incubated together at room temperature for 2 hours with slow rotation to allow adhesion. Approximately five beads became attached to each cell and there was some clumping of attached and unattached beads. Iodoacetamide, 2 μl of 1.0M solution, was then added to alkylate free sulphydryl groups. The samples were then spun down at 1500 g and the cells were lysed in HBSS, 0.075% SDS, 1% NP40, 0.2 mM AEBSF, for 4 minutes on ice. Beads were then isolated with a magnet and washed twice with lysis buffer. Disulphide-bonded complexes between cellular cadherin and the Fc-fusion protein were analysed by SDS-PAGE and immunoblotting for cadherin cytoplasmic domain as described above. To ensure equal loading, membranes were stripped and re-assayed with anti-Human Fc.

### Atomic Force Imaging

N-cadherin-Fc was purified using Protein A Sepharose as previously described (Corps et al., 2001). Samples were centrifuged at 100,000 g for 45 minutes to remove any aggregates and diluted to 1 μg/ml in 5 mM HEPES, 150 mM NaCl, 5 mM CaCl2, pH7.5, supplemented with 5 mM NiCl2. A volume of 50 μl was pipetted onto freshly cleaved mica (Goodfellow, Huntingdon, UK) and incubated at room temperature for 10 minutes. Unattached protein was then washed away with the same buffer. The protein molecules were then examined in the presence of 30 μl fresh buffer. AFM imaging was performed using a Nanoscope IIIa Multimode atomic force microscope (Veeco/Digital Instruments, Santa Barbara, Calif.) equipped with a J scanner. The N-cadherin-Fc molecules were imaged using oxide-sharpened silicon nitride probes (DNP-S; Digital Instruments) with a spring constant of 0.32 N/m operating in tapping mode at a drive frequency of ˜7-9 kHz.

### 1.2 Results

### Conformation of Dimeric N-Cadherin-Fc Fusion Protein

Electron microscopic examination of cadherin molecules as pentamers fused to cartilage oligomeric matrix protein (COMP) or as dimers linked to immunoglobulin-Fc, has revealed ‘ring’ and ‘spectacle-like’ structures deemed to represent cis- and trans-(adhesive) dimerisation, respectively (Ahrens et al., 2003; Pertz et al., 1999). As a first step in the current series of experiments, we examined our preparation of N-cadherin-Fc protein by atomic force microscopy (AFM) in the presence of 5 mM calcium to see whether similar structures were detectable. All wild type molecules had a Y-shaped form with the cadherin domains curving away from each other and the Fc region clearly distinguishable as a structure forming the ‘stem’ of the Y (FIG. 1). No N-cadherin domain 1 interactions were seen. The orientation of the curved cadherin domains suggests that the contact surfaces for dimerisation, identified in crystal structures (Boggon et al., 2002; Pertz et al., 1999), could not easily be juxtaposed, and it is possible that the Fc hinge region in our construct imposes mechanical constraints which militate against dimerisation of the N-terminal cadherin domains. Although we cannot completely rule out the possibility that contact with the mica substratum may have disrupted some dimers, the results suggest that at least a majority of the molecules in our preparations had this Y shape. The absence of adhesive dimers (spectacle-like structures) previously seen by electron microscopy in cadherin-Fc preparations (Ahrens et al., 2003) could be attributable to technical differences. Our preparation did not contain glycerol and the protein concentration used to coat the mica in our experiments was approximately 100-fold lower than that used for previous electron microscopy studies.

### Relationship Between Trp2 and Interdomain Calcium in the Structural Integrity of N-Cadherin Domain 1

A polyclonal antibody, K7, was prepared against a synthetic linear peptide in the βB strand of N-cadherin domain 1 (FIG. 2). Efficient antibody binding required that the peptide epitope be released from structural constraints of the domain fold. K7 was tested against N-cadherin-Fc containing or lacking the mutation W2G or the junctional mutation D134A which prevents co-ordination of the third calcium atom, Ca3, in the EC1-EC2 junction (Nagar et al., 1996). The two mutations were tested singly or in combination in an assay containing 1.25 mM calcium. Alternatively, calcium was added to the assay at varying levels. Results are shown in FIG. 3. In FIGS. 3a and 3b the N-cadherin-Fc used was the normal dimeric form as depicted in FIG. 1. FIG. 3a shows that antibody K7 failed to bind to wild type N-cadherin or to the D134A mutant, and reactivity with the W2G protein was low. Nevertheless, the two mutations in combination gave a strongly positive result. In FIG. 3b, the single mutation W2G was tested at a range of calcium levels. Antibody binding decreased to background as the calcium concentration reached 0.75 mM. These results suggest that calcium and Trp2 act in conjunction to stabilise the structure of domain 1. An alternative explanation could be that calcium-dependent interactions occur between the two cadherin units in the dimeric fusion protein which could prevent access of the antibody to the K7 epitope. Despite indications from our AFM scans that such interactions do not occur in our preparations, we tested monomeric N-cadherin-Fc to eliminate this possibility (FIG. 3c). In these molecules, dimerisation via Fc had been prevented by mutations in the CH3 domain.

The titrations were closely similar to those in FIG. 3a, showing that epitope masking could not explain our results. Because Trp2 is not part of the peptide epitope, the structural effect of this amino acid is almost certainly attributable to its integration into a hydrophobic pocket in the domain structure, as previously observed by NMR (Haussinger et al., 2004) and X-ray crystallography (Pertz et al., 1999).

### Antibody GC4 Detects Docking of Trp2

The epitope for a hitherto uncharacterized commercial N-cadherin antibody, GC4 (Volk and Geiger, 1984), was mapped using a panel of N-cadherin-Fc fusion proteins bearing point mutations (FIGS. 4a,b). The epitope was found to include residues K64, P65, D67 and Q70. Mapping results were similar in the presence or absence of calcium. The GC4 epitope lies opposite the βA strand, far distant from Trp2. Binding of GC4 was prevented by the mutation W2G, regardless of the presence (FIG. 5a) or absence (FIG. 5b) of calcium. This is persuasive evidence that GC4 binding requires Trp2 to be docked into the domain structure. Antibody binding to wild type N-cadherin was greater in the absence of calcium (compare FIGS. 5a,b,c). This could be due to modulation of Trp2 docking by calcium, with low levels of calcium favouring integration of Trp2. Alternatively, calcium may have a local effect on the epitope or its accessibility; both propensities could operate. To explore this issue further, we tested the hydrophobic pocket mutation A78M, which would hinder, but not necessarily preclude, Trp2 docking. This mutation is known to inhibit adhesion (Tamura et al., 1998). FIG. 5d shows that A78M inhibited binding of GC4 in the presence of 1.25 mM Ca2+, compared to wild type.

The result is consistent with impaired Trp2 docking. In contrast, when calcium was removed, both wild type and the A78M mutant gave equally high binding (FIG. 5e). This supports the explanation that calcium modulates Trp2 docking. In an alternative strategy to compromise insertion of Trp2 into the hydrophobic pocket, the N-terminus of Ncadherin was extended by three amino acids, MDP.

An extension would be expected to have a negative effect on integration of Trp2 into the domain (Haussinger et al., 2004) but, again, would not necessarily preclude docking (Pertz et al., 1999; Schubert et al., 2002). In keeping with results for the A78M mutation, the MDP mutation strongly inhibited binding of GC4 in the presence of calcium (FIG. 5f). As with the A78M mutant, removal of calcium from the MDP extension mutant restored binding of GC4 to levels obtained with the wild type molecule (FIG. 5g). We next showed that the effect of calcium can be attributed to its co-ordination in the EC1-EC2 junction. The mutation D134A, which disrupts co-ordination of Ca3 in this position, increased binding of GC4, compared with wild type (FIG. 5h), despite the presence of calcium in the assay buffer.

The result was similar with the MDP extension mutant (FIG. 5i), showing that the D134A junctional mutation had the same effect as removing calcium from the medium. Because GC4 binding requires Trp2 to be docked, regardless of calcium, these results argue strongly that calcium modulates a dynamic equilibrium that exists between docked and undocked Trp2 so that depletion of calcium favours more stable integration of Trp2 into the domain structure.

### Trp2 Cross-Intercalation (Strand Exchange) is a Primary Event in Cadherin-Mediated Cell Adhesion.

Elegant structural studies have demonstrated cadherin dimerisation by strand exchange (Boggon et al., 2002; Haussinger et al., 2004; Shapiro et al., 1995). The question remains whether this happens in a physiological context between opposing cadherin molecules during cell adhesion. Strand exchange would orientate the molecules so that specific amino acids near Trp2 and its hydrophobic pocket are brought into close apposition. We reasoned that if complementary cysteine point mutations were located in these positions they should generate a ‘reporter’ disulphide bond during cell adhesion, if strand exchange occurs. Using the strand exchange structure PDB 1NCI (Shapiro et al., 1995), we modelled formation of two alternative disulphide bonds in these circumstances using the complementary mutations D1C-D27C (FIG. 6a) and D1C-R25C (FIG. 6b). The bonds could be formed in silico equally well using other strand exchange structures (Boggon et al., 2002; Haussinger et al., 2004) and also when Trp2 was docked into its own domain (Pertz et al., 1999; Schubert et al., 2002).

To test for the formation of disulphide bonds during cell adhesion, K562 Cells were transfected with N-cadherin bearing a single cysteine point mutation and allowed to adhere, in reducing conditions, to an assay plate coated with monomeric N-cadherin-Fc bearing the complementary mutation. Oxidising conditions were then restored and the formation of a disulphide bond between cellular cadherin and cadherin-Fc was detected by immunoblotting. Initially, essential parameters of the experimental strategy were validated by testing adhesion of N-cadherin^{+ve} DX3 melanoma cells to N-cadherin-Fc molecules bearing the cysteine point mutations.

Monomeric N-cadherin-Fc, mutated to prevent Fc-Fc interaction, was used for most of our experiments in order to avoid the complication of disulphide bonded dimerisation in the hinge region of the Fc-fusion protein. FIG. 7 establishes that monomeric and dimeric N-cadherin-Fc support adhesion of DX3 Cells equally well throughout a range of coating concentrations. It is possible that cis-dimerisation of the monomer may occur at the highest coating levels but this could not happen as the monomer is diluted out on the plate. FIG. 8a shows that the single mutations, D1C, R25C and D27C had little or no effect on adhesion of DX3 Cells in normal (oxidising) adhesion buffer, but the double mutants D1C,R25C and D1C,D27C abolished adhesion completely. It is to be expected that in these molecules Trp2 would be ‘locked’ into its own domain by an adjacent disulphide bond and would be unavailable for strand exchange. Reducing conditions largely restored the function of the double mutants (FIG. 8b).

FIG. 9 shows matched expression of chicken N-cadherin mutant proteins by the K562 transfectants used for the present experiments (FIG. 9a) and demonstrates that untransfected K562 Cells lacked natural expression of human N-cadherin (FIG. 9b). Strong expression of human N-cadherin by DX3 Cells is also shown. Assurance that K562 Cells do not naturally express any classical cadherin was obtained by western blotting using pan cadherin antibody, which gave negative results (not shown). FIG. 9c Compares the molecular size of cellular cadherin from the transfectants with that of the monomeric Fcfusion protein and shows that the cellular material has a slightly higher molecular size than the fusion protein.

The K562 transfectants were allowed to adhere to N-cadherin-Fc monomer bearing complementary or non-complementary cysteine mutations. Trans-dimers were detected by immunoblotting, either for cellular cadherin using pan cadherin antibody to the cytoplasmic domain (FIG. 10a), or for the fusion protein using anti-Fc (FIG. 10b). The results show that disulphide-bonded trans-dimers formed only when complementary cysteine mutations were apposed, i.e. D1C-R25C or D1C-D27C (FIGS. 10a,b, upper panels). In addition to trans-dimers, D1C-D1C and D27C-D27C cis-homodimers formed both on the cells and in the coating layer of Fc-fusion protein on the assay plate (indicated in FIG. 10). This did not happen with the R25C mutation. The trans-dimers, which consisted of N-cadherin-Fc monomer linked to a molecule of cellular N-cadherin, were distinguishable from cellular cis-dimers or N-cadherin-Fc homodimers by molecular size. The size difference is clear in FIG. 10a, but less so in FIG. 10b. Here, formation of a trans-dimer is seen clearly with the combination D1C-R25C (left panel), but is not apparent with D1C-D27C (adjacent track). A weak trans-dimer band in this case would be largely obscured by the strong cis-dimer signal. The N-cadherin-Fc cisdimers which formed on the assay plate are seen in the right hand panel. The major bands running slightly above 97 kDa in all the blots represent cadherin molecules that either have not formed an adhesive contact or have failed to produce the disulphide bond. Lower panels in FIGS. 10a,b show results from running the samples under reducing conditions, where dimer bands are not detectable.

A second protocol was used to test for the formation of the disulphide bond in trans-alignment. It was designed to avoid detection of cis-dimers. In this strategy, mutant Ncadherin-Fc (the conventional fusion protein dimerised via Fc) was coated to magnetic beads which were allowed to adhere to the K562 transfectants. The cells were then lysed and the beads were separated from the lysate to extract the disulphide-bonded species in trans-alignment from the remainder of the cellular cadherin. FIG. 11 (left panel) shows results using K562 transfectants expressing wild type N-cadherin or the three cysteine mutations adhering to beads coated with N-cadherin-Fc fusion protein bearing the mutation D27C. The blot was developed using antibody to cadherin cytoplasmic domain. As before, the disulphide-bonded species formed only with the complementary pair D1CD27C, giving a major band at approximately 300 kDa. This represents one molecule of dimeric Fc-fusion protein, approximately 200 kDa, disulphide-bonded to one molecule of cellular cadherin. Other bands were also present representing higher order assemblies. The lower panel shows a loading control and the right hand panel shows monomeric cellular cadherin, for comparison. These results taken together provide persuasive evidence that strand exchange occurs during cell adhesion. Further, the observation that disulphide-bonded homodimers between molecules bearing the same cysteine mutation, D1C or D27C, are produced in cis-, but not trans-, orientation argues that the molecular alignments here must differ from those which form the adhesive dimer.

### 1.3 Discussion

In this example we have used two antibodies to investigate the stability of domain 1 in relation to the roles of calcium and Trp2 and have demonstrated a major effect of both factors acting in concert. The data complements recent NMR studies (Haussinger et al., 2004) and provides a perspective that is not available from crystal structures. It is possible that antibody binding could itself direct conformational change, but this would be subject to the varying constraints imposed by calcium and Trp2 in our experiments and, therefore, would not affect our conclusions. The published crystal structures of cadherins all show a full complement of calcium atoms in the domain 1-2 junction, with or without intercalation of Trp2 into the domain structure. The α-carbon backbone is closely similar in all cases. In contrast, the original NMR structure of domain 1 of Ecadherin (Overduin et al., 1995) shows neither intercalated Trp2 nor correctly coordinated calcium atoms, and here the α-carbon trace shows significant displacement compared with that in the crystal structures. Our present data suggest that this NMR structure would be relatively unstable and the βB strand readily displaced from the domain. Results with the peptide antibody K7 show that Trp2 and calcium act in concert to stabilise domain 1, each by separate means limiting flexibility of the βB strand and constraining the overall conformation.

Binding of antibody GC4 showed an absolute requirement for Trp2, regardless of the presence or absence of calcium. Because this amino acid is located on the opposite side of the domain, 30 Å away from the GC4 epitope, the result argues persuasively that reactivity with GC4 requires Trp2 to be located in the hydrophobic cavity in domain 1. In these circumstances Trp2 would impose structural constraints on the GC4 epitope, either via the core of the domain or by limiting movement of the βA strand at its base. Our data show that reduction of calcium in the domain 1-2 junction increased GC4 binding. The effect was modest with wild type N-cadherin but greater with the mutant A78M or the N-terminally extended version MDP; each of these modifications would compromise intercalation of Trp2. The results taken together argue persuasively that calcium modulates the dynamic equilibrium between docked and undocked Trp2. Thus, at low calcium levels Trp2 is more firmly integrated than at physiological levels.

Dimerisation by strand exchange requires that Trp2 swaps from insertion in its own domain to that of its neighbour, overcoming an activation barrier (Haussinger et al., 2004). The present results are consistent with recent NMR data showing that calcium facilitates this process (Haussinger et al., 2004). Our interpretation of the effect of calcium predicts that dimerisation by strand exchange requires calcium but, once formed, the dimer can be isolated from the cell surface in buffers lacking calcium. This is consistent with empirical evidence (Chitaev and Troyanovsky, 1998; Klingelhofer et al., 2002).

Our data with GC4 reflect intramolecular docking of Trp2 rather than strand exchange because we obtained closely similar results (not shown) with monomeric N-cadherin-Fc over a wide titration range where, at lower coating levels, N-cadherin monomers would be widely spaced on the assay plate. In this example we did not test whether GC4 detects crossintercalation of Trp2, as well as intramolecular docking. It is notable that our cell adhesion experiments demonstrate that monomeric N-cadherin coated to an assay plate, over a range of concentrations, supports cell adhesion equally as efficiently as the normal fusion protein dimerised at the IgG heavy chain hinge region. This dispels a widely held view that cis-dimerisation is an obligatory stage in the formation of the adhesive complex (Brieher et al., 1996; Ozawa, 2002; Takeda et al., 1999; Tomschy et al., 1996). Recently, Troyanovsky et al. (Troyanovsky et al., 2003) used a bifunctional sulphydryl cross-linking reagent to determine the orientation of cadherin molecules in cis- and trans-dimers and concluded that a strand exchange mechanism provided the best explanation for both types of dimer. The present example addresses this issue by a more direct strategy. The formation of a disulphide bond during cell adhesion using complementary, but not identical, cysteine point mutations on opposing cadherin molecules provides compelling evidence for strand exchange. This degree of specificity in the formation of the bond demands that during adhesion Trp2 is either inserted into the hydrophobic pocket of the opposing cadherin molecule or is poised very close to it. By similar reasoning, the cis-dimers we detected between adjacent cadherin molecules bearing the same mutation, D1C or D27C, could not be formed by the mutual strand exchange mechanism depicted in current crystal structures (Boggon et al., 2002; Haussinger et al., 2004; Shapiro et al., 1995). This does not rule out the possibility that strand-exchange cis-dimers may occur on the cell surface; they would not be disulphide-linked and would escape detection on our gels. It is important to emphasise that disulphide bonded cis-dimers were formed with the D1C and D27C mutations, but not with the R25C mutation. This demonstrates that these bonds were not a consequence of random contacts between cadherin molecules. Specificity of the bond for D1C and D27C limits the possible orientations that the molecules can adopt in making the cis-contact. A favourable orientation to achieve this discrimination is for adjacent cadherin molecules to be aligned in parallel, similar to the calcium-dependent C2-symmetric E-cadherin dimer recently determined by NMR (Haussinger et al., 2002). Alternatively, cross-intercalation of one Trp2 residue, as opposed to mutual exchange, may allow sufficient rotation of the domains to bring two D27C mutations into apposition. This arrangement can be seen in a hypothetical structure (PDB 1Q5C) for the orientation of desmosomal cadherins, based on electron tomography (He et al., 2003).

The present example provides the most direct evidence so far that strand exchange is a primary event in cadherin-mediated cell adhesion. This conclusion must be reconciled with three controversial outstanding issues, viz, the questions of cadherin type-specificity (Klingelhofer et al., 2000; Niessen and Gumbiner, 2002; Nose et al., 1990), the role of the conserved HAV motif (Makagiansar et al., 2001; Renaud-Young and Gallin, 2002; Williams et al., 2000; Williams et al., 2002) and the contribution of domains 2-4 to cell adhesion (Chappuis-Flament et al., 2001; Zhu et al., 2003). We envisage that Trp2 exchange is the initial event in cadherin-mediated adhesion; the HAV motif is not directly involved and the interaction is not cadherin type-specific. Subsequently, secondary interactions that require other regions of the cadherin molecule follow. These contacts facilitate clustering, provide specificity or initiate intracellular signalling. We suggest that our present strategy of using ‘reporter’ disulphide bonds to reveal adhesive surfaces in a physiological setting may be a powerful tool to investigate these interactions.

### 2. Example 2

### Modulation of Cadherin Adhesion

Example 1 provides compelling evidence that the mutual “strand exchange” (also known as “βA strand exchange”) mechanism is a primary event in cadherin mediated cell adhesion. In essence, the process depends entirely on formation an ionic bond (salt bridge) between cadherin molecules on opposing cells. The bond is formed between the N-terminal NH2 group on Asp1 in domain EC1 of one cadherin molecule and the acidic side chain of Glu89 on EC1 of the opposing molecule (FIG. 18).

It is known that correct post-translational processing of cadherin molecules is essential for adhesion (Ozawa and Kemler, 1990). Type I cadherins are synthesised with a prodomain of more than 100 amino acids which has the structure of a typical cadherin fold (Koch et al., 2004). There is an unstructured linker of approximately 30 amino acids between the prodomain and the first domain, EC1, of the mature molecule. A multi-basic recognition motif is cleaved by furin proteases to give the mature cadherin molecule which has a conserved typtophan as the second amino acid from the N-terminus. Failure to remove the prodomain prevents adhesion (Koch et al., 2004; Ozawa and Kemler, 1990). The presence of even a few additional amino acids at the N-terminus completely ablates adhesive function (Corps et al., 2001; Ozawa and Kemler, 1990). In keeping with this observation, a recent NMR study (Haussinger et al., 2004) showed that correct processing at the N-terminus was required for the strand exchange mechanism or for intramolecular docking of Trp2 into its own domain. The crystal structure of C-cadherin, in which the N-terminus is correctly processed, shows that strand exchange brings the amino group of Asp 1 in close proximity to the acidic side chain of a conserved amino acid, Glu 89, in the opposing cadherin domain, suggesting that a salt bridge could form here to stabilise Trp2 docking (Boggon et al., 2002). However, the significance of the putative salt bridge has been questionable because crystal structures of E- and N-cadherins show Trp2 integrated into the domain fold despite extension of the N-terminus and, consequently, the absence of this ionic bond (Pertz et al., 1999; Schubert et al., 2002; Shapiro et al., 1995).

In the present example we have investigated the significance of the salt bridge in cell adhesion mediated by N-cadherin. We have prevented formation of the bond in one or both components of the adhesive dimer by extending the N-terminus or by mutating Glu 89 to alanine. The results demonstrate with striking clarity that the salt bridge plays a vital role in adhesion by stabilising Trp 2 docking and that intramolecular and intermolecular docking of Trp 2 are in dynamic equilibrium. When the E89A mutation and the N-terminal extension are present in opposing cadherin molecules respectively, they form a complementary pair, each preventing intramolecular docking of Trp 2 but facilitating strand exchange in one direction. In these circumstances the normal equilibrium is disturbed and the strength of cadherin adhesion is greatly increased. Therefore, by manipulating this salt bridge by mutagenesis we can completely ablate or enhance strand exchange and hence modulate cadherin-mediated cell adhesion. The inhibitory activity of a well-known commercial antibody to mouse N-cadherin (NCD2) is now understandable because its epitope is directly adjacent to the E89 salt bridge. By targeting this salt bridge or the flexible hinge at the base of the βA strand, inhibition or enhancement of adhesion may be achieved, for example by drugs. The present invention therefore provides a rational basis for the design of drugs which will inhibit or enhance cell adhesion in all solid tissues, including skin, gut, blood vessels, organs and solid tumours. Because cadherin-mediated recognition is also central to the development of neuronal networks in the brain and to the function of the neuronal synapse, such drugs may be useful in therapy for neurodegenerative disease. It is important to note that the salt bridge mechanism which we have elucidated is more widespread in the cadherin superfamily than the HAV recognition motif mentioned above. We provide here that antibodies, proteins, peptides or natural or synthetic organic compounds could be used to interfere with the salt bridge or the flexible hinge at the base of the βA strand which regulates strand exchange.

### 2.1 Materials and Methods

Preparation of DNA Constructs and their Transfection into Cell Lines

Mutations were prepared in full length chicken N-cadherin cDNA in pcDNA3.1 using the QuikChange mutagenesis method (Stratagene). Constructs were stably transfected into K562 lymphomyeloid cells as described in Example 1 above. These cells have been shown to lack natural expression of cadherins (see Example 1). Clonal cell lines were obtained by limiting dilution and selected for equal expression of N-cadherin. Wild type and mutant chicken N-cadherin Fc fusion proteins containing the five extracellular domains were prepared, standardised and quantitated as described in Example 1. All cell lines were cultured in DMEM+10% FCS containing G418 at 1 mg/ml.

### Cadherin-Mediated Cell Adhesion

Adhesion tests were conducted substantially as described in Example 1. Briefly, K562 Cells or L cells transfected with wild type or mutant N-cadherin were allowed to settle for 45 minutes at 37° C. onto N-cadherin Fc fusion proteins coated at 1 μg/ml to a 96 well plate. Non-adherent cells were then washed off and residual adherent cells were quantitated by measuring acid phosphatase activity. Assays were conducted in quadruplicate and results are expressed as % cells adhering +/−SEM.

### Bead Aggregation Assay

Dynabeads (Dynal Biotech) coupled to Protein A were coated with N-cadherin Fc at 1 μg/ml in calcium and magnesium-free HBSS containing 0.1% Tween 20, 1% FCS and 4 mM EDTA. Eppendorf tubes containing beads and fusion protein were rotated slowly for 1 hour at room temperature to allow binding to take place. The beads were then washed in the above assay buffer lacking EDTA and then resuspended in the same buffer supplemented with 1.25 mM CaCl₂. Beads were allowed to aggregate in a volume of 100 μl for 2 hours at 37° C. by slow rotation, in an eppendorf tube, at approximately 20 rpm. Aggregation was then assessed by light microscopy.

### Immunofluorescent Staining of K562 Transfectants

Cells were stained for chicken N-cadherin using antibody NCD-2 (R & D Systems) at 5 μg/ml. The secondary antibody was FITC-labelled goat anti-rat IgG (Serotec, UK). For staining transfectants with N-cadherin Fc fusion proteins, the cells were treated with the fusion proteins at 5 μg/ml for 90 minutes on ice in Hanks Balanced Salt Solution (HBSS) containing 2% FCS and 0.1% sodium azide. After washing, bound fusion protein was detected with FITC-labelled goat anti-human Fc (Serotec) and quantitated by flow cytometry using a FACSCalibur (Becton Dickinson).

### Cleavage of the N-Cadherin Prodomain

L cells expressing mouse N-cadherin with an uncleaved prodomain (see Koch et al., 2004) were obtained from Dr Weisong Shan (Montreal Neurological Institute). The normal furin cleavage site, RQKR, had been replaced with a Factor Xa site, IEGR, to give the correct N-terminus after digestion. Trypsin also cleaved at this position (Koch et al., 2004) and proved to be more efficient than Factor Xa. L cells suspended in HBSS containing 0.1% BSA were treated with 0.01% trypsin (Sigma, Type XI) in the presence of 2 mM Ca²⁺ for 10 minutes at 37° C. and the digestion was then quenched with soyabean trypsin inhibitor, 0.5 mg per ml (Sigma, Type I-S). Cells were then washed and tested for adhesion to N-cadherin Fc-fusion protein. To check for complete removal of the prodomain, the cells were lysed in SDS sample buffer and the cadherin analysed by SDS PAGE under reducing conditions on a 4-12% gradient gel. N-cadherin was identified by western blotting using rabbit anti-pan cadherin antiserum specific for the cytoplasmic domain (Sigma, code C3678) followed by affinity-purified HRP-labelled sheep anti-rabbit IgG, F(ab′)₂-specific (Serotec).

### Viewing Molecular Structures

Cadherin structures were displayed using Swiss PDB Viewer (http://www.expasy.org/spdbv/).

### 2.2 Results

### Disrupting the Salt Bridge Between the N-Terminus and Glu 89

FIG. 12a shows the position of the two salt bridges formed during mutual strand exchange. To prevent formation of this bond, Glu 89 of N-cadherin was mutated to alanine or, alternatively, the N-terminus was extended by adding two glycine residues to Asp 1 to displace the N-terminal amino group away form the acidic side chain of Glu 89. Mutant N-cadherin proteins were expressed in K562 lymphomyeloid cells which were matched for equal cell surface expression of the respective cadherins (FIG. 12b). Transfectants were tested for adhesion to mutant or wild type N-cadherin Fc fusion proteins (FIG. 13). Disruption of the salt bridge by either the E89A mutation or the double glycine N-terminal extension strongly inhibited adhesion to wild type N-cadherin. Similarly, each of the two mutants failed to adhere to its own kind. In sharp contrast, the combination of the E89A mutation on one side of the adhesive pair with the GG extension on the other resulted in markedly stronger adhesion than that given by wild type N-cadherin, a high proportion of the cells becoming flattened to the assay plate. The double mutation E89A plus the GG extension in the same molecule prevented adhesion in all circumstances. A negative control to demonstrate that the assay reflected cadherin-mediated adhesion was provided by the N-cadherin Fc mutant D134A. This mutation prevents co-ordination of the third calcium atom, Ca3, in the junction between domains 1 and 2 and is known to prevent cadherin-mediated adhesion (Corps et al., 2001; Ozawa et al., 1990). The D134A mutation alone (FIG. 13a) or in combination with E89A or with the GG N-terminal extension (FIG. 13b) reduced adhesion to background levels. The enhanced adhesion seen with the complementary pair of mutants E89A and the GG extension is further illustrated in FIG. 13c, using a range of concentrations of N-cadherin Fc coated to the assay plate. Adhesion of cells bearing the E89A mutation to N-cadherin-Fc having the GG extension was reversed by chelation of calcium (FIG. 18). The reverse combination where cells expressing the GG mutation adhered to N-cadherin-Fc bearing the mutation E89A behaved similarly.

### Affinity Between the E89A and GG Extension Mutants

To investigate whether the enhanced adhesion observed with this complementary pair reflects an increase in affinity, we tested whether soluble N-cadherin Fc fusion proteins would bind to cell surface N-cadherin using a protocol similar to immunofluorescent staining by antibodies. FIG. 14 shows that binding between wild type N-cadherin molecules was undetectable. In contrast, the interaction between the E89A mutant and the GG extension mutant gave strong cell surface staining, approaching that seen with antibodies to cadherins. Previous studies (Baumgartner et al., 2000; Haussinger et al., 2004) measuring affinity between normal trans-acting cadherin molecules have reported K_{D} values in the range 10⁻³ to 10⁻⁵M. Although we have not yet obtained precise measurements of affinity between the complementary cadherin mutants, the present data clearly shows a large increase in affinity compared with that between wild type molecules.

### Effect of an Uncleaved Prodomain

In order to investigate whether an uncleaved prodomain has the same effect as a two amino acid extension to the N-terminus, we tested the ability of L cell transfectants expressing unprocessed N-cadherin to adhere to the E89A mutant. FIG. 15a shows strong adhesion of the transfectants to the E89A Fc fusion protein but no adhesion to wild type N-cadherin or to the D134A negative control. A Factor Xa cleavage site introduced into the transfected N-cadherin allowed removal of the prodomain from the cell surface with either Factor Xa or trypsin. Trypsin proved to be more efficient, almost completely removing the prodomain (FIG. 15b). In these circumstances the cells acquired the ability to adhere to wild type N-cadherin while adhesion to the E89A mutant was greatly reduced (FIG. 15c).

### Model for Strand Exchange

An explanation for our results is given in FIG. 16. Panel (a) shows N-cadherin domain 1 in isolation. As shown in Example 1, there is a dynamic equilibrium between docked and un-docked Trp 2 which favours the docked form. Insertion of Trp 2 into its hydrophobic acceptor pocket is stabilized by the salt bridge between Glu 89 and the N-terminus. By analogy with other 3D domain swap systems (Bergdoll et al., 1997), it is possible that the hinge loop at the base of the βA strand may be under spring-like tension, perhaps imposed by Pro 6 and neighbouring amino acids. Panel (b) shows the strand-swapped dimer. For strand exchange to occur, an activation barrier must be surmounted as Trp2 leaves the hydrophobic pocket (Haussinger et al., 2004). A transition state, in which Trp 2 is un-docked, is sampled from both sides and is depicted in the interface as an ‘unshaded’ tryptophan. Adhesion between the two domains is moderate. The difference in free energy between the ‘closed’ monomer in which Trp2 is docked into its own domain and the strand-swapped dimer is small because the #A strand enjoys the same contacts in each, the two forms differing only in the angle adopted by the hinge loop. In (c), extension of the N-terminus on one side de-stabilises both intramolecular and intermolecular docking of Trp 2 on that strand. Mutual strand exchange between the two domains cannot take place. Cross docking of Trp2 on the wild type strand is possible but this competes unfavourably with intramolecular docking into its own, wild type, domain. Thus, adhesion is very weak. Similar logic applies in (d) where the E89A mutation prevents intramolecular docking on that side. Again, strand exchange cannot be mutual. The strand from the E89A mutant can cross to the wild type domain but cross docking competes with intramolecular docking of Trp 2 on the wild type side. In (e) the two mutations form a complementary pair. The activation barrier for strand donation by the E89A mutant is greatly reduced because the salt bridge can form only in the trans position. Intramolecular docking on either side is denied, so cross docking of Trp2 into the pocket of the GG mutant is unimpaired. Even though only one strand is exchanged, adhesion is enhanced compared with the wild type interaction. In FIG. 16(f), when the double mutation is present in both partners, the salt bridge is prevented on either side and adhesion is completely lost. Finally, in FIG. 16(g)-(i) further examples of complementary pairs of cadherin molecules in which intramolecular docking is inhibited but intermolecular is favoured, causing strong adhesion between the molecules (see also below).

### Removing Trp2 or Blocking the Hydrophobic Pocket

In the experiments described so far in this example, disruption of the salt bridge prevented intramolecular docking of Trp 2 in one or both components of the dimer. In an alternative strategy to prevent intramolecular docking, we removed Trp2 by introducing the mutation W2G or blocked the hydrophobic acceptor pocket with an isoleucine side chain projecting into the cavity using the mutation A801. N-cadherin Fc fusion proteins with these mutations were then tested against our panel of K562 transfectants (FIG. 17a). In keeping with the explanation given in FIG. 5, the W2G mutant acted as a strand acceptor and therefore adhered strongly to the E89A mutant whereas the A80I mutant was a strand donor and adhered to the GG extension mutant. To determine whether the W2G mutant and the A80I mutant adhered strongly to one another, as would be predicted, the proteins were coated separately to dynabeads and the two types were then mixed and tested for cadherin-dependent aggregation, FIG. 17b shows that the mixed preparation of beads formed large clumps, aggregating more strongly than beads coated with wild type N-cadherin. In contrast, beads coated with the W2G or A801 mutants and tested separately clustered in twos and threes, whereas beads coated with the negative control mutant, D134A, were entirely monodisperse. To test whether all five extracellular domains of N-cadherin (EC1-EC5) are necessary for the strong adhesive interaction between the complementary mutants, we compared the adhesive activity of the W2G mutant prepared as a full length (EC1-EC5) Fc fusion protein with that of the same mutant prepared as a truncated construct containing only cadherin domains EC1 and EC2. Results in FIG. 19 show that the two domain construct was almost as efficient as the full length cadherin in supporting adhesion.

### 2.3 Discussion

Example 2 demonstrates decisively that the strand exchange mechanism of cadherin adhesion requires formation of a salt bridge between opposing cadherin molecules involving the N-terminus of one molecule and E89 of the other. The bond is a major feature of the free energy landscape that governs strand exchange. A second factor is the hydrophobic interaction between Trp 2 and its acceptor pocket. For stable adhesion, the energy contributions of both factors are required. In addition, a hydrogen bond formed between the amide nitrogen of Val 3 and the carbonyl oxygen of residue 25 may also contribute to the stability of Trp 2 intercalation (Haussinger et al., 2004). Formation of the cadherin adhesive dimer can be regarded as a relatively uncomplicated example of the 3D domain swap mechanism for protein oligomerization (Bennett and Eisenberg, 2004; Rousseau et al., 2003). A high energy barrier must be overcome as the structural component to be exchanged is released from its own domain and becomes available for exchange, but the difference in free energy between the monomer and the dimer is small. In our experiments, intramolecular docking of Trp 2 was prevented in both components of the cadherin dimer by disrupting the salt bridge. This greatly reduced the energy barrier for strand exchange. By using a complementary pair of mutations, the GG N-terminal extension on one side and E89A on the other, the energetics were changed strongly to favour exchange of one strand. These mutants behaved as “molecular Velcro™” forming a strongly adhesive complementary pair but neither adhering to its own kind. It is likely that the effect of the GG extension in this context was solely to displace the N-terminus away from the acidic side chain of E89 to prevent formation of the salt bridge. This is corroborated by our observations (data not shown) that alternative short extensions to the N-terminus, e.g. Met-Asp-Pro or a single Cys, had a similar complementary effect with the E89A mutant (see FIG. 16). Strong adhesion was observed even with unprocessed N-cadherin, though titrations suggest that the prodomain had an inhibitory effect compared with an extension of only two amino acids. Some steric hindrance by the prodomain would be expected because the long flexible linker (Koch et al., 2004) would allow freedom of movement of the uncleaved domain to interfere with the strand exchange process.

Our results were obtained with N-cadherin, a classical cadherin. On the basis of multiple alignment of amino acid sequences of non-classical cadherins and structural modelling by the present inventors and others (see May et al., 2005), the present invention also provides that non-classical (Type II) cadherins, desmocollins and desmogleins all have a similar strand exchange mechanism dependent on a salt bridge in the position described here. In the protocadherin family, N-terminal peptide analysis suggests that protocadherins alpha also have a conserved tryptophan as the second amino acid (Gevaert et al., 2003), indicating that the same mechanism may apply in this group also. Variations of the strand swap model are therefore proposed according to the present invention to apply throughout the whole cadherin family.

To explain cadherin type-specificity by the strand exchange mechanism, we propose according to the present invention that optimal adhesion between wild type cadherins may require free energy changes accompanying mutual strand exchange to be equal on both sides of the adhesive dimer. At least two factors influence the energy landscape, the N-terminal salt bridge and the hydrophobic interaction between Trp 2 and its pocket. The former would be affected by the electrostatic environment in the vicinity of Glu 89 and the latter by non-conserved amino acids lining the hydrophobic pocket. Our experiments are consistent with the hypothesis of energy balance and implicate both factors in the specificity displayed by N- and E-cadherins.

The second cadherin domain, EC2, is required for correct co-ordination of calcium in the junction between the first cadherin domain, EC1, and EC2, and the disruptive effect of the D134A mutation in the present experiments demonstrate that, in this respect, EC2 is essential for strand exchange. Our results do not rule out the possibility that EC3 or EC4 Could provide additional contact sites or be involved in other ways. Assays used in prior art studies to test for adhesive contacts involving inner domains have varied greatly in sensitivity and results must be interpreted accordingly. The cell adhesion test in the present example is very robust and is unlikely to reveal weak interactions. In contrast, our bead aggregation assay is more sensitive and it is notable that the W2G mutant and the A80I mutant which, individually, could not undergo strand exchange by tryptophan docking showed weak but detectable aggregation when tested separately. The result reflect the presence of one or more additional contact sites, for example not located on EC1 or EC2, which are also provided according to the present invention. It is pertinent to observe that formation of the intermolecular salt bridge between E89 and the N-terminus is likely to require correct angular alignment of opposing N-terminal domains. The presence of the inner domains may facilitate optimal orientation, indeed, the curvature of the complete extracellular region may be significant in this respect.

The present invention offers new insights into the strand exchange mechanism. The observation that cadherin affinity can be greatly increased by lowering activation energy using salt bridge mutations provides in one aspect a rational basis for designing alternative strategies for modulating, for example greatly increasing, cadherin adhesion.

The foregoing examples are meant to illustrate the invention and do not limit it in any way. One of skill in the art will recognize modifications within the spirit and scope of the invention as indicated in the claims.

### REFERENCES

Ahrens, T. et al. (2003) J. Mol. Biol. 325, 733-42.
Ahrens, T. et al. (2002) J. Biol. Chem. 277, 19455-60.
Alattia, J. R. et al. (1997) FEBS Lett. 417, 405-8.
Baumgartner, W. et al. (2000) Proc. Natl. Acad. Sci. USA 97, 4005-10.
Bennett, M. J. and Eisenberg, D. (2004) Structure (Camb) 12, 1339-41.
Bergdoll, M. et al. (1997) Structure 5, 391-401.
Boggon, T. J. et al. (2002) Science 296, 1308-13.
Brieher, W. M. et al. (1996) J. Cell Biol. 135, 487-96.
Cavallaro, U. and Christofori, G. (2004) Nat Rev Cancer 4, 118-32.
Chappuis-Flament, S. et al. (2001) J. Cell Biol. 154, 231-43.
Chitaev, N. A. and Troyanovsky, S. M. (1998) J. Cell Biol. 142, 837-46.
Corps, E. et al. (2001) J. Biol. Chem. 276, 30862-70.
Corps, E. M. et al. (2003) Eur. J. Immunol. 33, 2599-608.
Dall'Acqua, W. et al. (1998) Biochemistry 37, 9266-73.
Gevaert, K. et al. (2003) Nature Biotechnology 21, 566-9.
Haussinger, D. et al. (2004) EMBO J. 23, 1699-708.
Haussinger, D. et al. (2002) J. Mol. Biol. 324, 823-839.
He, W. et al. (2003) Science 302, 109-13.
Hirano, S. et al. (2003) Front. Biosci. 8, d306-55.
Kitagawa, M. et al. (2000) Biochem. Biophys. Res. Commun. 271, 358-63.
Klingelhofer, J. et al. (2000) J. Cell Sci. 113 (Pt 16), 2829-36.
Klingelhofer, J. et al. (2002) Mol. Cell. Biol. 22, 7449-58.
Koch, A. W. et al. (2004) Structure (Camb) 12, 793-805.
Laur, O. Y. et al. (2002) Arch. Biochem. Biophys. 400, 141-7.
Makagiansar, I. T. et al. (2001) Pharm. Res. 18, 446-53.
May, C. et al. (2005) Blood 105, 4337-44.
Miyaguchi, K. (2000) J. Struct. Biol. 132, 169-78.
Nagar, B. et al. (1996) Nature 380, 360-4.
Niessen, C. M. and Gumbiner, B. M. (2002) J. Cell Biol. 156, 389-399.
Nose, A. et al. (1990) Cell 61, 147-55.
Overduin, M. et al. (1995) Science 267, 386-9.
Ozawa, M. (2002) J. Biol. Chem. 277, 19600-8.
Ozawa, M. and Kemler, R. (1990) J. Cell Biology 111, 1645-50.
Pertz, O. et al. (1999) EMBO J. 18, 1738-47.
Pokutta, S. et al. (1994) Eur. J. Biochem. 223, 1019-26.
Puch, S. et al. (2001) J. Cell Sci. 114, 1567-77.
Renaud-Young, M. and Gallin, W. J. (2002) J. Biol. Chem. 277, 39609-16.
Rousseau, F. et al. (2003) Structure (Camb) 11, 243-51.
Sambrook et al. (1989) Molecular Cloning: A Laboratory Manual, Cold Spring Harbor Laboratory Press, New York.
Schubert, W. D. et al. (2002) Cell 111, 825-36.
Shapiro, L. et al. (1995) Nature 374, 327-37.
Sivasankar, S. et al. (2001) Biophys. J. 80, 1758-68.
Takeda, H. et al. (1999) Nat. Struct. Biol. 6, 310-2.
Tamura, K. et al. (1998) Neuron 20, 1153-63.
Thiery, J. P. (2003) Curr. Opin. Genet. Dev. 13, 365-71.
Tomschy, A. et al. (1996) EMBO J. 15, 3507-14.
Troyanovsky, R. B. et al. (2003) Mol. Cell. Biol. 23, 7965-72.
Volk, T. and Geiger, B. (1984) EMBO J. 3, 2249-60.
Wheelock, M. J. and Johnson, K. R. (2003) Annu. Rev. Cell Dev. Biol. 19, 207-35.
Williams, E. J. et al. (2000) Mol. Cell. Neurosci. 15, 456-64.
Williams, G. et al. (2002) J. Biol. Chem. 277, 4361-7.
Zhu, B. et al. (2003) Biophys. J. 84, 4033-42.

All references cited are incorporated herein by reference.

1. A pair of cadherin molecules modified to enhance intermolecular adhesion compared with corresponding unmodified cadherin molecules.

2. A pair of cadherin molecules according to claim 1, in which intermolecular adhesion is enhanced by reducing or eliminating intramolecular binding within each cadherin molecule.

3. A pair of cadherin molecules according to claim 2, in which intramolecular binding is reduced or eliminated by diminishing or preventing intramolecular binding of an N-terminal binding strand of each cadherin molecule with a binding strand acceptor pocket of the each cadherin molecule.

4. A pair of cadherin molecules according to claim 2, in which intramolecular binding is reduced or eliminated by diminishing or preventing the formation of an intramolecular ionic bond between the NH₂ terminus of each cadherin molecule with a contact acidic amino acid residue of each cadherin molecule.

5. A pair of cadherin molecules according to claim 1, in which intermolecular adhesion is facilitated by binding of an N-terminal binding strand of one cadherin molecule with a binding strand acceptor domain of the other cadherin molecule.

6. A pair of cadherin molecules according to claim 1, in which intermolecular adhesion is facilitated by an ionic bond between a contact acidic amino acid residue of one cadherin molecule and the NH₂ terminus of the other cadherin molecule.

7. A pair of cadherin molecules according to claim 1, in which the cadherin molecules are modified by altering the primary structure of each cadherin molecule.

8. A pair of cadherin molecules according to claim 1, in which the cadherin molecules are modified by contacting one or both cadherin molecules with one or more substances which enhance intermolecular adhesion and/or reduce or eliminate intramolecular binding.

9. A pair of cadherin molecules according to claim 3, in which the N-terminal binding strand is derived from or equivalent to the βA strand of the ECI domain of mature wild-type human N-cadherin, or a functional equivalent thereof.

10. A pair of cadherin molecules according to claim 3, in which the binding strand acceptor pocket is derived from or equivalent to the hydrophobic Trp2 acceptor pocket in the βA strand of the EC1 domain of mature wild-type human N-cadherin, or a functional equivalent thereof.

11. A pair of cadherin molecules according to claim 4, in which the contact acid amino acid residue is derived from or equivalent to Glu89 of mature wild-type human N-cadherin, or a functional equivalent thereof.

12. A pair of cadherin molecules according to claim 4, in which the ionic bond is a salt bridge.

13. A pair of polypeptides which adhere to each other with an affinity greater than that between mature wild-type human N-cadherin molecules.

14. A pair of cadherin molecules according to claim 1, in which each cadherin molecule or each polypeptide is a functional fragment, equivalent, homologue or variant of mature wild-type human N-cadherin.

15. A pair of cadherin molecules according to claim 1, in which each cadherin molecule or each polypeptide has at least 80% or greater homology with mature wild-type human N-cadherin or a functional fragment, equivalent, homologue or variant of mature wild-type human N-cadherin.

16. A pair of cadherin molecules according to claim 1, in which each cadherin molecule or each polypeptide has at least 80% or greater homology with mature wild-type human N-cadherin or a functional fragment, equivalent, homologue or variant of mature wild-type human N-cadherin excluding the transmembrane and/or cytoplasmic domains of mature wild-type human N-cadherin.

17. A method of adhering a pair of polypeptides such as cadherin molecules by intermolecular adhesion, comprising the step of contacting the polypeptides or cadherin molecules as defined in claim 1, thereby allowing intermolecular adhesion.

18. A method of increasing adhesion between two cadherin molecules, comprising reducing or eliminating intramolecular binding within each cadherin molecule and allowing formation of an ionic bond between an acidic amino acid residue of one cadherin molecule and the NH₂ terminus of the other cadherin molecule.

19. A method of claim 18, in which intermolecular adhesion is facilitated by binding of an N-terminal binding strand of one cadherin molecule with a binding strand acceptor domain of the other cadherin molecule.

20. A substance which modulates intramolecular binding of one or more cadherin molecules by reducing or enhancing intermolecular adhesion between the molecules, wherein the substance excludes antibodies.

21. A method for using a substance which modulates intramolecular binding of one or more cadherin molecules for reducing or enhancing intermolecular adhesion between the molecules.

22. A method for screening a candidate compound for the ability to modulate cadherin-mediated cell adhesion, comprising contacting the pair of cadherin molecules according to claim 1, in the presence and absence of the candidate compound and thereby evaluating the ability of the candidate compound to modulate cadherin-mediated cell adhesion.

23. A method of increasing adhesion between a first cell and a second cell, comprising contacting the pair of cadherin molecules according to claim 1, when one of the pair is attached to the first cell and the other of the pair is attached to the second cell.

24. An isolated nucleic acid molecule encoding the pair of cadherin molecules according to claim 1.

25. A pair of isolated nucleic acid molecules in which each nucleic acid molecule encodes one of the pair of cadherin molecules according to claim 1.

26. A host cell comprising of the pair of cadherin molecules according to claim 1.

27. A kit comprising of the pair of cadherin molecules according to claim 1.

28. A solid substrate having at least one surface with a coating thereon, the coating comprising or consisting of modified cadherin molecules which are one of the pair of modified cadherin molecules as defined in claim 1.

29. A solid substrate having at least one surface with a coating thereon, the coating comprising or consisting of polypeptide molecules which are one of the pair of polypeptide molecules as defined in claim 13.

30. A solid substrate according to claim 28 wherein the substrate is a plate or bead.

31. A method according to claim 17 wherein the intermolecular adhesion step occurs by contacting the molecules in the presence of a liquid medium containing free calcium ions, and wherein in a further step the intermolecular adhesion is reversed by depletion or removal of free calcium ions in the medium.

32. A pair of polypeptides according to claim 13, in which each cadherin molecule or each polypeptide has at least 80% or greater homology with mature wild-type human N-cadherin or a functional fragment, equivalent, homologue or variant of mature wild-type human N-cadherin.

33. A pair of polypeptides according to claim 13, in which each cadherin molecule or each polypeptide has at least 80% or greater homology with mature wild-type human N-cadherin or a functional fragment, equivalent, homologue or variant of mature wild-type human N-cadherin excluding the transmembrane and/or cytoplasmic domains of mature wild-type human N-cadherin.

34. A method for screening a candidate compound for the ability to modulate cadherin-mediated cell adhesion, comprising contacting the pair of polypeptides according to claim 13, in the presence and absence of the candidate compound and thereby evaluating the ability of the candidate compound to modulate cadherin-mediated cell adhesion.

35. A method of increasing adhesion between a first cell and a second cell, comprising contacting the pair of polypeptides according to claim 13, when one of the pair is attached to the first cell and the other of the pair is attached to the second cell.

36. An isolated nucleic acid molecule encoding the pair of polypeptides according to claim 13.

37. A pair of isolated nucleic acid molecules in which each nucleic acid molecule encodes one of the pair of polypeptides according to claims 13.

38. A host cell consisting of the pair of polypeptides according to claims 13.

39. A host cell consisting of the isolated nucleic acid molecule according to claim 24.

40. A host cell consisting of the pair of isolated nucleic acid molecules according to claim 25.

41. A kit consisting of the pair of polypeptides according to claim 13.

42. A kit consisting of the isolated nucleic acid molecule according to claim 24.

43. A kit consisting of the host cell according to claim 26.

44. A pair of polypeptides according to claim 13, in which each cadherin molecule or each polypeptide is a functional fragment, equivalent, homologue or variant of mature wild-type human N-cadherin.
